SEQUENCE LISTING (8 sequences)

SEQ ID NO: 1 - protein, 732 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGATAPGKKRPVEQSPQEPDSSSGIGKTGQQPAKKRLNFGQTGDSESVPDPQPLGEPPATPAAVGPTTASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 4 aa, Artificial Sequence
  Description of Artificial Sequence Synthetic peptide
SEER

SEQ ID NO: 3 - protein, 8 aa, Artificial Sequence
  Description of Artificial Sequence Synthetic peptide
TPGGNATR

SEQ ID NO: 4 - protein, 10 aa, Artificial Sequence
  Description of Artificial Sequence Synthetic peptide
DLDPKATEVE

SEQ ID NO: 5 - DNA, 54 bp, Artificial Sequence
  Description of Artificial Sequence Synthetic primer
  modified_base (25)..(29): a, c, t, g, unknown or other
ccctacacgacgctcttccgatctnnnnncagaactcaaaatcagtccggaagt

SEQ ID NO: 6 - DNA, 58 bp, Artificial Sequence
  Description of Artificial Sequence Synthetic primer
  modified_base (33)..(37): a, c, t, g, unknown or other
gactggagttcagacgtgtgctcttccgatctnnnnngccaggtaatgctcccatagc

SEQ ID NO: 7 - DNA, 22 bp, Artificial Sequence
  Description of Artificial Sequence Synthetic primer
ccttcgcttcaaaaaatggaac

SEQ ID NO: 8 - DNA, 24 bp, Artificial Sequence
  Description of Artificial Sequence Synthetic primer
aaaagcactctgattgacaaatac

(19) United States
(12) Patent Application Publication
(10) Pub. No.: US 2021/0032660 A1
(43) Pub. Date: Feb. 4, 2021

(54) LIVER TROPIC RECOMBINANT AAV6 VECTORS THAT EVADE NEUTRALIZATION

(71) Applicants: ASKLEPIOS BIOPHARMACEUTICAL, INC., Research Triangle Park, NC (US); UNIVERSITY OF FLORIDA RESEARCH FOUNDATION, INCORPORATED, Gainesville, FL (US)

(72) Inventors: R. Jude SAMULSKI, Chapel Hill, NC (US); Mavis AGBANDJE-MCKENNA, Gainesville, FL (US); Antonette BENNETT, Gainesville, FL (US)

(73) Assignees: ASKLEPIOS BIOPHARMACEUTICAL, INC., Research Triangle Park, NC (US); UNIVERSITY OF FLORIDA RESEARCH FOUNDATION, INCORPORATED, Gainesville, FL (US)

(21) Appl. No.: 17/042,543

(22) PCT Filed: Mar. 28, 2019
(86) PCT No.: PCT/US2019/024567

§ 371 Date: Sep. 28, 2020

Related U.S. Application Data
(60) Provisional application No. 62/649,691, filed on Mar. 29, 2018.

(51) Int. Cl. C12N 15/86; C07K 14/005

(52) U.S. Cl. CPC C12N 15/86; C12N 2750/14143; C12N 2750/14122; C07K 14/005

(57) ABSTRACT

As demonstrated herein, a modified recombinant AAV6 vector is provided that transduces the liver and has reduced neutralization of transduction of liver by ADK6 antibody. Accordingly, embodiments of the invention relate to liver tropic rAAV6 vectors that evade neutralization.

### CROSS REFERENCE TO RELATED APPLICATIONS

This international application claims benefit under 35 U.S.C. § 119(e) of U.S. Provisional Application No. 62/649,691 filed Mar. 29, 2018, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 25, 2019, is named 046192-091920WOPT_SL.txt and is 8,294 bytes in size

### FIELD OF INVENTION

Embodiments of the invention relate to liver tropic rAAV6 vectors that evade neutralization.

### BACKGROUND

Adeno-associated virus (AAV) is a non-pathogenic dependent parvovirus that is used as a vector for gene therapy. Multiple serotypes and variants have been identified, and several have been used in clinical trials. Although therapeutic effect of AAV vectors has been achieved in clinical trials, one of the major challenges of AAV vectors is the necessity of treating naïve subjects, those not previously exposed to the virus, and the inability to redose the vector due to immune response. Accordingly, there is a need in the art for the development of gene therapy vectors that can evade immune responses, and that can efficiently deliver and express genes in specific tissues.

### SUMMARY

In one aspect of the invention a modified recombinant AAV6 vector is provided that comprises a substitution at one or more amino acid residues selected from the group consisting of S264, G266, N269, H272, Q457, S588 and T589 corresponding to AAV6 VP1 numbering, wherein the rAAV6 vector transduces the liver and has reduced neutralization of transduction of liver by ADK6 antibody as compared to the rAAV6 vector lacking the one or more substitutions. In one embodiment, the modified AAV6 vector also comprises a lysine (K) at amino acid 531 (AAV6 VP1 numbering). In one embodiment, the modified AAV6 vector comprises an arginine (R) at amino acid 531 corresponding to AAV6 VP1 numbering. In certain embodiments where a K or R at amino acid 531 is present, at least two, at least three, at least four, at least five, at least six, or at least 7 of the one or more amino acids are substituted. In certain embodiments of the above, the one or more substitutions comprise conserved amino acid substitutions. In certain embodiments, the one or more substitutions comprise non-conserved substitutions.

In one embodiment described herein, the modified rAAV6 vector further comprises one or more modified regions of amino acids selected from the group consisting of: 262-272; 382-386, 445-457, 459, 469-473, 488-489, 494-496, 499-515, 571-579, 584-589, and 593-595, corresponding to AAV6 VP1 numbering, and wherein at least one or more amino acids in the one or more modified regions are substituted. SEQ ID NO: 1 set forth AAV6 VP1. Thus S264, G266, N269, I-1272, Q457, S588, T589 and K or R 531 corresponding to AAV6 VP1 are S264, G266, N269, H272, Q457, S588, T489, K531 and R531 of SEQ ID NO: 1, respectively.

In another aspect, a modified rAAV6 vector comprising one or more modified regions of amino acids selected from the group consisting of: 262-272; 382-386, 445-457, 459, 469-473, 488-489, 494-496, 499-515, 571-579, 584-589, and 593-595 (corresponding to AAV6 VP1 numbering) is provided, where at least one or more amino acids in the one or more modified regions are substituted, and wherein the rAAV6 vector transduces the liver and has reduced neutralization of transduction of liver by ADK6 antibody as compared to the rAAV6 vector lacking the one or more substitutions.

In another, a modified rAAV6 vector comprising one or more modified regions of amino acids selected from the group consisting of: 269-272; 445-450; 469-471; 493; 501-515; 584-587 (corresponding to AAV6 VP1 numbering) is provided, where at least one or more amino acids in the one or more modified regions are substituted, and wherein the rAAV6 vector transduces the liver and has reduced neutralization of transduction of liver by ADK6 antibody as compared to the rAAV6 vector lacking the one or more substitutions.

In some embodiments, in any of the aspects described herein, the modified rAAV6 vector comprises K531, or R531.

In some embodiments of each of the aspects described herein, the modified rAAV6 vector further comprises a substitution of one or more amino acids that bind to sialic acid selected form the group consisting of: N447, S472, V473, N500, T502, and W503.

In some embodiments, in each of the aspects described herein, the modified rAAV6 vector comprises amino acid substitution at one or more amino acid regions selected from the group consisting of: 456-459; 492-499; and 588-597.

In some embodiments, in each of the aspects described herein, the modified rAAV6 vector comprises one or more of the amino acid sequences selected from the group consisting of: SEER at 456-499 (SEQ ID NO: 2), TPGGNATR (SEQ ID NO: 3) at 492-499; DLDPKATEVE (SEQ ID NO: 4) at 588-597.

In some embodiments, in each of the aspects described herein, the modified rAAV6 vector comprises the vector has reduced neutralization of liver transduction by human anti-sera to unmodified rAAV6 as compared to neutralization of the unmodified rAAV6 vector.

In some embodiments, in each of the aspects described herein, the modified rAAV6 vector comprises the vector has reduced neutralization of liver transduction by mouse anti-sera to unmodified rAAV6 as compared to neutralization of the unmodified rAAV6 vector.

In some embodiments, in each of the aspects described herein, the modified rAAV6 vector comprises the vector has reduced neutralization of liver transduction by rhesus macaques anti-sera to unmodified rAAV6 as compared to neutralization of the unmodified rAAV6 vector.

In another aspect, a method for isolating an AAV6 virion that retains liver tropism and that has reduced neutralization by ADK6 antibody is provided. The method comprises: generating a saturation mutagenesis AAV6 library wherein each of the amino acids selected from the group consisting of S264, G266, N269, H272, Q457, S588 and T589, are substituted with each of the 20 different natural or unnatural amino acid and in any combination of all or fewer than all positions; and performing rounds of evolution by infecting liver cells (e.g. hepatocytes) or tissue with the rAAV6 library; and screening for reduced neutralization by ADK6 or wild-type rAAV6 anti-sera. In one embodiment of the above, the rAAV6 of the library comprises either K531 or R531. In one embodiment, the method further comprises screening for the loss of sialic acid binding (e.g. by column chromatography). In one embodiment, the method further comprises screening for the presence of sialic acid binding (e.g. by column chromatography).

In another aspect, a method for identifying an AAV6 virion that retains liver tropism and exhibits reduced neutralization by an AAV6 neutralizing antibody such as ADK6 antibody is provided. The method comprises generating a saturation mutagenesis library of one or more modified regions of amino acids selected from the group consisting of: 262-272; 382-386, 445-457, 459, 469-473, 488-489, 494-496, 499-515, 571-579, 584-589, and 593-595, wherein the one or more regions are substituted with each of the 20 different natural or unnatural amino acid and in any combination of all or fewer than all positions; and performing rounds of evolution by infecting liver cells (e.g. hepatocytes) or tissue with the rAAV6 library; and screening for reduced neutralization by ADK6 or anti-sera to wild type rAAV6. In one embodiment the rAAV6 of the library comprises either K531 or R531. The modified rAAV6 vectors exhibit reduced neutralization by ADK6 antibody and significantly transduce the liver. In certain embodiments, the modified rAAV6 vectors has reduced neutralization of liver transduction by human antisera to unmodified rAAV6 (e.g. wild-type virus) as compared to the neutralization of unmodified rAAV6 vector. In certain embodiments, the modified rAAV6 vectors has reduced neutralization of liver transduction by antisera of other species (mouse, Rhesus, dog etc.) to unmodified rAAV6 (e.g. wild-type virus) as compared to the neutralization of unmodified rAAV6 vector.

Also provided are methods for delivering a transgene to a subject comprising administering the modified rAAV6 vectors described herein.

### BRIEF DESCRIPTION OF FIGURES

FIGS. 1A-1E. Cryo-EM and image reconstruction of the AAV6-ADK6 FAb complex. FIG. 1A. Example cryo-electron micrograph of AAV6-ADK6 capsid-FAb complex. Projections from the capsid surface indicate FAb decoration. FIG. 1B. Fourier Shell Correlation plotted against resolution (FSC plot) for the AAV6-ADK6 structure. The estimated resolution is 16 and 13 Å at FSC 0.5 and 0.143, respectively. FIG. 1C. Surface density representation of the cryo-reconstructed AAAV6-ADK6 Fab complex structure viewed along the icosahedral 2-fold axis. The capsid density is depicted as well as the FAb. Right: Zoomed in image of the complex density map and docked pseudo-atomic model of the capsid (grey) and FAb (pink). Residue K531 is shown in blue and labeled. FIG. 1D. AAV6 capsid surface image (in gray) with FAb predicted contact residues in hot pink, occluded residues (as defined in the text) in light pink, and K531 and L584, within the occluded region, in blue (basic) and yellow (hydrophobic), respectively. The viral asymmetric unit, bounded by a 5-fold and two 3-fold axes intercepted by a 2-fold axis, is depicted by a large black triangle. The 2-, 3-, and 5-fold axes are represented by an oval, triangle, and pentagon, respectively. FIG. 1E. 2D “roadmap” projection of the residues within a viral asymmetric unit. The images were generated by the Chimera (Pettersen et al., 2004), PyMol(Schrödinger, 2017), and RIVEM programs (Xiao and Rossmann, 2007) for panels FIGS. 1C-1E, respectively.

FIGS. 2A-2C. Production and purification of WT and variant AAV1 and AAV6 vectors. FIG. 2A. Residue positions and type for surface amino acids plus internal residue 642 which differ between AAV1 and AAV6. FIG. 2B. Negative stained EM of WT and variant AAV1 and AAV6 vectors. The scale bar is shown in white on the first EM image. FIG. 2C. Quantitation of purified vector genome titer determined by qPCR for WT and variant AAV1 and AAV6 vectors.

FIGS. 3A-3C. ADK6 binding and neutralization assays. FIG. 3A. Immunoblot of denatured WT and variant AAV1 and AAV6 vectors detected by B1 (which recognizes a linear epitope of the C-terminus of the VP1/2/3 common region) (top) and by ADK6 (which recognizes native capsids). ADK6 interacts specifically with AAV6, and this interaction is lost in mutant AAV6-K531E and restored in AAV1-E531K. FIG. 3B and FIG. 3C. Neutralization assays for WT and variants of AAV1 and AAV6, respectively, in the presence of ADK6. Transduction is normalized to the luciferase signal for WT virus at zero antibody concentration.

FIGS. 4A-4B. The functional surface of AAV6. FIG. 4A and FIG. 4B. Capsid surface and 2D “roadmap” projection of AAV6, respectively. The SIA and HS receptor binding residues are shown for basic, polar, and hydrophobic residues, respectively. The viral asymmetric unit are depicted as in FIG. 1. The images for panels FIG. 4A and FIG. 4B were generated in the PyMol (Schrödinger, 2017) and RIVEM programs (Xiao and Rossmann, 2007), respectively.

### DESCRIPTION

Embodiments of the present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of +/−20%, +/−10%, +/−5%, +/−1%, +/−0.5%, or even +/−0.1% of the specified amount.

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

As used here, “systemic tropism” and “biodistribution” indicate that the virus capsid or virus vector of the invention exhibits tropism for and can transduce tissues throughout the body (e.g., brain, lung, skeletal muscle, heart, liver, kidney and/or pancreas). In certain embodiments of the invention, systemic transduction of the central nervous system (e.g., brain, neuronal cells, etc.) is observed. In other embodiments, systemic transduction of cardiac muscle tissues is achieved. In certain embodiments herein, the modified vector may exhibit a different systemic tropism, or a different biodistribution profile, and exhibit a selective tropism with enhanced transduction in specific tissues as compared to the parent virus that has not been modified. In certain embodiments, the biodistribution profile is substantially the same, however there is an enhancement of transduction in certain tissues that receive the vector.

As used herein, “selective tropism” or “specific tropism” means delivery of virus vectors to and/or specific transduction of certain target cells and/or certain tissues. In one embodiment, the modified vectors exhibit a selective tropism that differs form the parent virus.

Unless indicated otherwise, “efficient tropism,” or similar terms, can be determined by reference to a suitable control (e.g., at least about 50%, 60%, 70%, 80%, 85%, 90%, 95%, 100%, 125%, 150%, 175%, 200%, 250%, 300%, 350%, 400%, 500% or more of the transduction or tropism, respectively, of the control (unmodified virus vector).

In certain embodiments, the modified vector “does not efficiently transduce” or “does not have efficient tropism” for a target tissue, or similar terms, by reference to a suitable control, such as unmodified parental vector. In particular embodiments, the virus vector does not efficiently transduce (i.e., has does not have efficient tropism) for liver, kidney, gonads and/or germ cells. In particular embodiments, undesirable transduction of tissue(s) (e.g., germ cells, liver etc.) is 90% less, or 80% less, or 60% less, or 50% less, 20% or less, 10% or less, 5% or less, the level of transduction of the desired target tissue(s) (e.g., skeletal muscle, diaphragm muscle, cardiac muscle and/or cells of the central nervous system, etc.).

As used herein, “transduction” of a cell by recombinant parvovirus refers to the transfer of genetic material into the cell by the incorporation of nucleic acid into parvovirus particle and subsequent transfer into the cell where DNA can be transcribed.

Unless indicated otherwise, “enhanced transduction” refers to an increase in transduction as by a statistically significant amount as compared to the parent rAAV vector prior to modifying the vector as indicated.

Similarly, unless indicated otherwise, by “reduced transduction” refers to a decrease in transduction by a statistically significant amount as compared to the parent rAAV vector prior to modifying the vector as indicated. “Reduced neutralization of transduction” refers to a reduced inhibition of transduction exhibited by a neutralizing antibody, e.g. ADK6, or ADK1, etc. The reduction is by a statistically significant amount as compared to the inhibition of transduction observed with unmodified rAAV6 in the presence of the neutralizing antibody, or e.g. antiserum. A reduced inhibition of transduction thus is an enhanced transduction in the presence of the neutralizing antibody as compared to the parent vector (unmodified) in the presence of the neutralizing antibody or particular anti-sera. Neutralization can be assessed by in vitro assays as described herein.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of treatment. In certain embodiments the prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject. In certain embodiments, the treatment is not curative.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

A “heterologous nucleotide sequence” or “heterologous nucleic acid” is a sequence that is not naturally occurring in the virus. In one embodiment, the heterologous nucleic acid comprises an open reading frame that encodes a therapeutic polypeptide. In one embodiment, the heterologous nucleic acid sequence is a noncoding DNA or RNA. In one embodiment, the heterologous nucleic acid encodes a nontranslated therapeutic RNA of interest, e.g. RNAi, miRNA, siRNA, anti-sense RNA, short hairpin or small hairpin RNA (shRNA), or ribozyme, or fragments thereof, or e.g. a sgRNA for CRISPER editing, or other gene editing molecule, e.g. ZFNs and TALENs. In some embodiments, the methods and compositions described herein, can comprise and/or be used to deliver CRISPRi (CRISPR interference) and/or CRISPRa (CRISPR activation) systems to a host cell. CRISPRi and CRISPRa systems comprise a deactivated RNA-guided endonuclease (e.g., Cas9) that cannot generate a double strand break (DSB).

CRISPRa can further comprise gRNAs which recruit further transcriptional activation domains. sgRNA design for CRISPRi and CRISPRa is known in the art (see, e.g., Horlbeck et al. eLife. 5, e19760 (2016); Gilbert et al., Cell. 159, 647-661 (2014); and Zalatan et al., Cell. 160, 339-350 (2015); each of which is incorporated by reference here in its entirety). CRISPRi and CRISPRa-compatible sgRNA can also be obtained commercially for a given target (see, e.g., Dharmacon; Lafayette, Colo.). Further description of CRISPRi and CRISPRa can be found, e.g., in Qi et al., Cell. 152, 1173-1183 (2013); Gilbert et al., Cell. 154, 442-451 (2013); Cheng et al., Cell Res. 23, 1163-1171 (2013); Tanenbaum et al. Cell. 159, 635-646 (2014); Konermann et al., Nature. 517, 583-588 (2015); Chavez et al., Nat. Methods. 12, 326-328 (2015); Liu et al., Science. 355 (2017); and Goyal et al., Nucleic Acids Res. (2016); each of which is incorporated by reference herein in its entirety.

As used herein, the terms “recombinant AAV (rAAV) vector” or “gene delivery vector” refer to a virus particle that functions as a nucleic acid delivery vehicle, and which comprises the vector genome (e.g., viral DNA [vDNA]) packaged within an AAV capsid. Alternatively, in some contexts, the term “vector” may be used to refer to the vector genome/vDNA alone.

A “rAAV vector genome” or “rAAV genome” is an AAV genome (i.e., vDNA) that comprises one or more heterologous nucleotide sequences. rAAV vectors generally require only the 145 base terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the minimal TR sequence(s) so as to maximize the size of the transgene that can be efficiently packaged by the vector. The structural and non-structural protein coding sequences may be provided in trans (e.g., from a vector, such as a plasmid, or by stably integrating the sequences into a packaging cell). The rAAV vector genome comprises at least one TR sequence (e.g., AAV TR sequence, synthetic, or other parvovirus TR sequence), optionally two TRs (e.g., two AAV TRs), which typically will be at the 5′ and 3′ ends of the heterologous nucleotide sequence(s), but need not be contiguous thereto. The TRs can be the same or different from each other.

The term “terminal repeat” or “TR” includes any viral terminal repeat and synthetic sequences that form hairpin structures and function as an inverted terminal repeat, such as the “double-D sequence” as described in U.S. Pat. No. 5,478,745 to Samulski et al. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Virol. 79: 5047-58), AAV5 (Walters et al., (2004) J. Virol. 78: 3361-71) and CPV (Xie et al., (1996) J. Mol. Biol. 6:497-520 and Tsao et al., (1991) Science 251: 1456-64).

An “AAV terminal repeat” or “AAV TR” may be from any AAV, including but not limited to serotypes 1, 2, 3, 4, 5, 6, 7, 8, 9, 10 or 11 or any other AAV now known or later discovered. The AAV terminal repeats need not have a wild-type terminal repeat sequence (e.g., a wild-type sequence may be altered by insertion, deletion, truncation or missense mutations), as long as at least one of the terminal repeat mediates the desired functions, a functional TR, e.g., replication, virus packaging, integration, and/or provirus rescue, and the like. One of skill in the art understands to choose a Rep protein that is functional for replication of the functional TR.

The modified rAAV6 vector used in methods of the invention can also be a “hybrid” parvovirus (e.g., in which the rAAV genome is from a different parvovirus or serotype than the AAV capsid protein described herein) e.g. as described in international patent publication WO 00/28004; WO 03/089612; and Chao et al., (2000) Molecular Therapy 2:619 (the disclosures of which are incorporated herein by reference in their entireties).

The modified virus vector used in methods of the invention can further be a duplexed parvovirus particle as described in international patent publication WO 01/92551 (the disclosure of which is incorporated herein by reference in its entirety). Thus, in some embodiments, double stranded (duplex) genomes carrying the heterologous nucleic acid can be packaged into the virus capsids of this invention.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used herein, the term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

Provided herein are modified rAAV6 vectors that comprise amino acid substitutions, e.g. in one or more modified regions of amino acids selected from the group consisting of: 262-272; 382-386, 445-457, 459, 469-473, 488-489, 494-496, 499-515, 571-579, 584-589, and 593-595 (amino acids of VP1 AAV6). The amino acid substitution/s in each of these regions can comprise substitution of all positions present in the region in any combination, or can comprises in any combination of fewer than all positions, e.g. resulting an amino acid sequence of X¹-X²-X³-X⁴-X⁵ . . . and so on, respectively (dependent upon the number of amino acids in the region), wherein X is any amino acid of the 20 amino acids other than the amino acid normally present at the position of SEQ ID NO: 1 (VP1 of wild-type AAV6).

Also provided herein are rAAV6 vectors that comprise amino acid substitutions, e.g. a substitution at one or more amino acid residues selected from the group consisting of S264, G266, N269, H272, Q457, S588 and T589 (AAV6 VP1 numbering), wherein the rAAV6 vector transduces the liver and has reduced neutralization of transduction of liver by ADK6 antibody as compared to the rAAV6 vector lacking the one or more substitutions. The modified rAAV6 vector can comprise substitution of each of these positions in any combination, or can comprises in any combination of fewer than all positions, e.g. resulting an amino acid sequence of X¹-X²—X³-X⁴-X⁵-X⁶-X⁷ . . . or less, e.g. X¹-X²-X³-X⁴-X⁵-X⁶; X¹-X²-X³-X⁴-X⁵; X¹-X²-X³-X⁴; X¹-X²-X³, X¹-X²; or X¹; wherein X is any amino acid other than the amino acid normally present at the at the position of SEQ ID NO: 1 (VP1 of wild-type AAV6).

Thus, the amino acid substitutions can be conserved, or can be non-conserved. In certain embodiments, the amino acid substitution can be of un-natural amino acids.

Virus capsids and rAAV6 viral vectors according to embodiments of the invention can be produced using any method known in the art, e.g., by expression from a baculovirus (Brown et al., (1994) Virology 198:477-488), or a mammalian cell.

The modifications to the rAAV6 capsid protein according to embodiments of the present invention are “selective” modifications. This approach is in contrast to previous work with whole subunit or large domain swaps between AAV serotypes (see, e.g., international patent publication WO 00/28004 and Hauck et al., (2003) J. Virology 77:2768-2774). In particular embodiments, a “selective” modification results in the substitution of less than about 10, 9, 8, 7, 6, 5, 4, 3, 2, or 3 contiguous amino acids in the specified regions, e.g. 262-272; 382-386, 445-457, 459, 469-473, 488-489, 494-496, 499-515, 571-579, 584-589, and 593-595 (amino acids of VP1 AAV6). In certain embodiments, at least 3, at least 4, at least 5, at least 6, at least 7, at least 8, at least 9, at least 10, or all of the amino acids in the specified region are substituted. In one embodiment, the amino acid region is selected form the group consisting of: 269-272; 445-450; 469-471; 493; 501-515; 584-587 (VP1 AAV6 numbering). Selected modification can include one or more specified amino acid regions, as well as can include one or more single amino acid substitutions at S264, G266, N269, H272, Q457, S588 and T589 (AAV6 VP1 numbering). As described herein, the selected modification of embodiments of the invention reduces neutralization by ADK6 antibody and retains significant transduction of the liver; it is liver tropic.

In one aspect of the invention a modified recombinant AAV6 vector is provided that comprises a substitution at one or more amino acid residues selected from the group consisting of S264, G266, N269, H272, Q457, S588 and T589 corresponding to AAV6 VP1 numbering, wherein the rAAV6 vector transduces the liver and has reduced neutralization of transduction of liver by ADK6 antibody as compared to the rAAV6 vector lacking the one or more substitutions. In one embodiment, the modified AAV6 vector also comprises a lysine (K) at amino acid 531 (AAV6 VP1 numbering). In one embodiment, the modified AAV6 vector comprises an Arginine® at amino acid 531 corresponding to AAV6 VP1 numbering. In certain embodiments where a K or R at amino acid 531 is present, at least two, at least three, at least four, at least five, at least six, or at least 7 of the one or more amino acids are substituted. In certain embodiments of the above, the one or more substitutions comprise conserved amino acid substitutions. In certain embodiments, the one or more substitutions comprise non-conserved substitutions.

In one embodiment described herein, the modified rAAV6 vector further comprises one or more modified regions of amino acids selected from the group consisting of: 262-272; 382-386, 445-457, 459, 469-473, 488-489, 494-496, 499-515, 571-579, 584-589, and 593-595, corresponding to AAV6 VP1 numbering, and wherein at least one or more amino acids in the one or more modified regions are substituted. SEQ ID NO: 1 set forth AAV6 VP1. Thus S264, G266, N269, I-1272, Q457, S588, T589 and K or R 531 corresponding to AAV6 VP1 are S264, G266, N269, H272, Q457, S588, T489, K531 and R531 of SEQ ID NO: 1, respectively.

In another aspect, a modified rAAV6 vector comprising one or more modified regions of amino acids selected from the group consisting of: 262-272; 382-386, 445-457, 459, 469-473, 488-489, 494-496, 499-515, 571-579, 584-589, and 593-595 (corresponding to AAV6 VP1 numbering) is provided, where at least one or more amino acids in the one or more modified regions are substituted, and wherein the rAAV6 vector transduces the liver and has reduced neutralization of transduction of liver by ADK6 antibody as compared to the rAAV6 vector lacking the one or more substitutions.

In another, a modified rAAV6 vector comprising one or more modified regions of amino acids selected from the group consisting of: 269-272; 445-450; 469-471; 493; 501-515; 584-587 (corresponding to AAV6 VP1 numbering) is provided, where at least one or more amino acids in the one or more modified regions are substituted, and wherein the rAAV6 vector transduces the liver and has reduced neutralization of transduction of liver by ADK6 antibody as compared to the rAAV6 vector lacking the one or more substitutions.

In some embodiments, in any of the aspects described herein, the modified rAAV6 vector comprises K531, or R531.

In some embodiments of each of the aspects described herein, the modified rAAV6 vector further comprises a substitution of one or more amino acids that bind to sialic acid selected form the group consisting of: N447, S472, V473, N500, T502, and W503.

The modified capsid proteins and capsids of the invention can further comprise any other modification, now known or later identified, e.g. a targeting peptide.

In some embodiments of this invention, the capsid protein, virus capsid or vector of this invention can have equivalent or enhanced transduction efficiency relative to the transduction efficiency of the AAV6 from which the capsid protein, virus capsid or vector of this invention originated. In some embodiments of this invention, the capsid protein, virus capsid or vector of this invention can have reduced transduction efficiency relative to the transduction efficiency of the AAV6 from which the capsid protein, virus capsid or vector of this invention originated. In some embodiments of this invention, the capsid protein, virus capsid or vector of this invention can have equivalent or enhanced tropism relative to the tropism of the AAV serotype from which the capsid protein, virus capsid or vector of this invention originated. In some embodiments of this invention, the capsid protein, virus capsid or vector of this invention can have an altered or different tropism relative to the tropism of the AAV6 from which the capsid protein, virus capsid or vector of this invention originated.

The modified rAAV vector used in methods of the invention further can include a heterologous nucleic acid (e.g. transgene, or gene editing nucleic acid) that has a therapeutic effect, with respect to a disease, when the heterologous nucleic acid is delivered to the of a subject. The therapeutic nucleic acid can be operably linked to a promoter (constitutive, cell-specific, or inducible). In this manner, the therapeutic nucleic acid can be produced in vivo in the subject. The subject can be selected for being in need of the therapeutic nucleic acid because the subject has a deficiency in the nucleic acid product, or because the production of the therapeutic nucleic acid in the subject imparts some therapeutic effect.

One of skill in the art can readily construct a rAAV vector carrying any therapeutic heterologous nucleic acid of interest using methods described herein and those well known to those of skill in the art (See example, Sambrook et al. (Molecular Cloning, A Laboratory Manual, Cold Spring Harbor Laboratory Press, New York 1989). The nucleotide and protein, polypeptide and peptide sequences for various genes have been previously disclosed, and may be found at computerized databases known to those of ordinary skill in the art. One such database is the National Center for Biotechnology Information's Genbank and GenPept databases. The coding regions for these known genes may be amplified, cloned into in the rAAV nucleic acid template disclosed herein. In one embodiment, the heterologous nucleic acid encodes a therapeutic polypeptide. Embodiments of the invention also relate to methods for the treatment or the prevention of disease by administration of the modified vectors.

The modified parvovirus vector may also comprise a heterologous nucleotide sequence that shares homology with and recombines with a locus on a host chromosome. This approach can be utilized, for example, to correct a genetic defect in a host cell. It will be understood by those skilled in the art that the heterologous nucleotide sequence(s) of interest may be operably associated with appropriate control sequences. For example, the heterologous nucleic acid may be operably associated with expression control elements, such as transcription/translation control signals, origins of replication, polyadenylation signals, internal ribosome entry sites (IRES), promoters, enhancers, and the like. Inducible expression control elements are preferred in those applications in which it is desirable to provide regulation over expression of the heterologous nucleic acid sequence(s). Inducible promoters/enhancer elements for gene delivery can be tissue-specific promoter/enhancer elements, and include smooth muscle specific (e.g. vascular smooth muscle cells and endothelial cell specific (e.g. vascular endothelial cells). Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter, FK506, or FK1012.

Vector Production

Methods for the production of recombinant vectors are well known to those of skill in the art. Except as otherwise indicated, standard methods known to the skilled artisan may be used for the construction of rAAV constructs, the modified capsid protein DNA sequences (cap), packaging vectors expressing the AAV rep and/or cap sequences, and transiently and stably transfected packaging cells. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York). As a further alternative, the modified rAAV-2 virus vectors of the invention can be produced in insect cells using baculovirus vectors to deliver the rep/cap genes and rAAV template as described, for example, by Urabe et al., (2002) Human Gene Therapy 13:1935-43.

For example, for rAAV, the rAAV vector can be produced by providing to a cell i) a nucleic acid template including the heterologous nucleic acid sequence and at least one TR sequence (e.g. AAV TR sequence) and ii) AAV sequences sufficient for replication of the nucleic acid template and encapsidation into AAV capsids (e.g., AAV rep sequences and AAV cap sequences that encode the modified AAV capsids described herein. The nucleic acid template can comprises at least one functional AAV ITR sequence, and replicate to two ITR's which are located 5′ and 3′ to the heterologous nucleic acid sequence(s), although they need not be directly contiguous thereto.

The nucleic acid template and AAV rep and cap sequences are provided under conditions (e.g. in the presence of helper functions, e.g. Ad, or HSV) such that recombinant virus vector comprising the nucleic acid template packaged within the modified parvoviral (e.g AAV or other parvovirus) capsid is produced within the cell. The virus vector can be collected from the medium and/or collected by lysing the cells.

Any suitable cell known in the art may be employed for production e.g. mammalian or insect, etc.

The TRs can be modified (e.g., truncated, mutated by substitution, deletion, addition, etc.) to impart different characteristics to a recombinant nucleic acid and/or to a virus vector of this invention. For example, non AAV terminal repeat sequences such as those of other parvoviruses (e.g., canine parvovirus (CPV), mouse parvovirus (MVM), human parvovirus B-19) that provide similar function for vector propagation, packaging, and transduction could be used in the vectors of this invention. In some embodiments of this invention, the TR can be modified with portions of non AAV terminal repeat sequences. In other embodiments of this invention, the TR can be substituted with non parvovirus terminal repeats such as an SV40 hair pin sequence that serves as the origin of SV40 replication. These represent only limited examples of modified TRs and other such modifications would be known to one of ordinary skill in the art.

Therapeutic Nucleic Acids

The modified vectors described herein can be used for the treatment of any disease that is susceptible to being treated or prevented by administering to a tissue a vector encoding a therapeutic heterologous nucleic acid of interest (e.g. therapeutic transgene or non-coding nucleic acid, DNA or RNA). Suitable transgenes for gene therapy are well known to those of skill in the art. For example, the altered vectors described herein can deliver transgenes and uses that include, but are not limited to, those described in U.S. Pat. Nos. 6,547,099; 6,506,559; and 4,766,072; Published U.S. Application No. 20020006664; 20030153519; 20030139363; and published PCT applications of WO 01/68836 and WO 03/010180, and e.g. miRNAs and other transgenes of WO2017/152149; each of which are hereby incorporated herein by reference in their entirety.

In one embodiment, the therapeutic transgene is a selected from the group consisting of: growth factors, interleukins, interferons, anti-apoptosis factors, cytokines, anti-diabetic factors, anti-apoptosis agents, coagulation factors, and anti-tumor factors, e.g. BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10(187A), viral IL-10, IL-11, IL-12, IL-13, IL-14, IL-15, IL-16 IL-17, and IL-18.

In one embodiment, the therapeutic heterologous nucleic acid is associated with a lack of expression or dysfunction of the gene. For example, listed are exemplary therapeutic transgenes and associated disease states; glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl Co A dehydrogenase, associated with medium chain acetyl Co A deficiency; ornithine transcarbamylase, associated with ornithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher disease; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppessor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes.

In one embodiment, the therapeutic heterologous nucleic acid is a genes associated with diseases or disorders associated with CNS; e.g. DRD2, GRIA1, GRIA2, GRIN1, SLClAl, SYP, SYT1, CHRNA7, 3Rtau/4rTUS, APP, BAX, BCL-2, GRIK1, GFAP, IL-1, AGER, associated with Alzheimer's Disease; UCH-L1, SKP1, EGLN1, Nurr-1, BDNF, TrkB, gstml, S 106p, associated with Parkinson's Disease; IT15, PRNP, JPH3, TBP, ATXN1, ATXN2, ATXN3, Atrophin 1, FTL, TITF-1, associated with Huntington's Disease; FXN, associated with Freidrich's ataxia; ASPA, associated with Canavan's Disease; DMD, associated with muscular dystrophy; and SMN1, UBE1, DYNC1H1 associated with spinal muscular atrophy.

In one embodiment, the therapeutic heterologous nucleic acid is a gene associated with diseases or disorders associated with the cardiovascular system, e.g. VEGF, FGF, SDF-1, connexin 40, connexin 43, SCN4a, HIF1a, SERCa2a, ADCY1, and ADCY6.

In one embodiment, the therapeutic heterologous nucleic acid is a gene associated with diseases or disorders associated with pulmonary system CFTR, A AT, TNFa, TGFpl, SFTPA1, SFTPA2, SFTPB, SFTPC, HPS 1, HPS 3, HPS 4, ADTB3A, ILIA, IL1B, LTA, IL6, CXCR1, and CXCR2.

In one embodiment, the therapeutic heterologous nucleic acid is a genes associated with diseases or disorders associated with the liver, e.g. al-AT, HFE, ATP7B, fumarylacetoacetate hydrolase (FAH), glucose-6-phosphatase, NCAN, GCKR, LYPLAL1, PNPLA3, lecithin cholesterol acetyltransferase, phenylalanine hydroxylase, and G6PC.

In one embodiment, the therapeutic heterologous nucleic acid is a gene associated with diseases or disorders associated with the kidney, e.g. PKD1, PKD2, PKHD1, NPHS 1, NPHS2, PLCE1, CD2AP, LAMB2, TRPC6, WT1, LMX1B, SMARCAL1, COQ2, PDSS2, SCARB3, FN1, COL4A5, COL4A6, COL4A3, COL4A4, FOX1C, RET, UPK3A, BMP4, SIX2, CDCSL, USF2, ROB02, SLIT2, EYA1, MYOG, SIX1, SIXS, FRAS 1, FREM2, GATA3, KALI, PAX2, TCF2, and SALL1.

In one embodiment, the therapeutic heterologous nucleic acid is a gene associated with diseases or disorders associated with the eye or ocular disease, e.g. VEGF, CEP290, CFH, C3, MT-ND2, ARMS2, TIMP3, CAMK4, FMN1, RHO, USH2A, RPGR, RP2, TMCO, SIX1, SIX6, LRP12, ZFPM2, TBK1, GALC, myocilin, CYP1B 1, CAV1, CAV2, optineurin and CDKN2B.

In one embodiment, the therapeutic heterologous nucleic acid is a gene associated with diseases or disorders associated with blood, e.g., red blood cells, e.g. Factor VIII (FVIII), Factor IX (FIX), von Willebrand factor (VWF).

In one embodiment, the therapeutic heterologous nucleic acid is a gene associated with apoptosis.

In one embodiment, the therapeutic heterologous nucleic acid is a tumor suppressor.

Accordingly, provided herein are methods for treating a disease comprising administering a modified vector as described herein that carrys a heterologous therapeutic nucleotide sequence. Non-limiting examples of disease to be treated include for example achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240). Additional exemplary diseases that can be treated by targeted integration include acquired immunodeficiencies, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

Pharmaceutical compositions of the present invention comprise an effective amount of one or more modified rAAV virus vector(s) or additional agent(s) dissolved or dispersed in a pharmaceutically acceptable carrier. The phrases “pharmaceutical or pharmacologically acceptable” refer to molecular entities and compositions that do not produce an adverse, allergic or other undesirable reaction, biological effect, when administered to an animal, such as, for example, a human, as appropriate.

The preparation of a pharmaceutical composition that contains at least one modified rAAV vector or additional active ingredient will be known to those of skill in the art in light of the present disclosure, as exemplified by Remington's Pharmaceutical Sciences, 18th Ed., Mack Printing Company, 1990, incorporated herein by reference. Moreover, for animal (e.g., human) administration, it will be understood that preparations should meet sterility, pyrogenicity, general safety and purity standards as required by the U.S. FDA Office of Biological Standards or equivalent governmental regulations in other countries, where applicable.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, and like materials and combinations thereof, as would be known to one of ordinary skill in the art (see, for example, Remington's Pharmaceutical Sciences, 18th Ed., Mack Printing Company, 1990, pp. 1289-1329, incorporated herein by reference). Except insofar as any conventional carrier is incompatible with the active ingredient, its use in the therapeutic or pharmaceutical compositions is contemplated.

A pharmaceutical composition comprising a modified rAAV vector and/or additional agent(s) may exploit different types of carriers depending on whether it is to be administered in solid, liquid or aerosol form, and whether it need be sterile for such routes of administration as injection. The pharmaceutical compositions can be administered intravenously, intradermally, intra-arterially, intra-graft, intraperitoneally, intralesionally, intracranially, intraarticularly, intraprostatically, intrapleurally, intratracheally, intranasally, intravitreally, intravaginally, intrarectally, topically, intratumorally, intramuscularly, intraperitoneally, subcutaneously, subconjunctivally, intravesicularly, mucosally, intrapericardially, intraumbilically, intraocularly, orally, topically, locally, inhalation (e.g. aerosol inhalation), injection, infusion, continuous infusion, localized perfusion bathing target cells directly (e.g., in an autogenous tissue graft), via a catheter, via lavage, in cremes, in lipid compositions (e.g., liposomes), or by any other method or any combination of the foregoing as would be known to one of ordinary skill in the art (see, for example, Remington's Pharmaceutical Sciences, 18th Ed., Mack Printing Company, 1990).

The modified vector and/or an agent may be formulated into a pharmaceutical composition in a free base, neutral or salt form. Pharmaceutically acceptable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as sodium, potassium, ammonium, calcium or ferric hydroxides; or such organic bases as isopropylamine, trimethylamine, histidine or procaine.

The practitioner responsible for administration will determine the concentration of active ingredient(s) in a pharmaceutical composition and appropriate dose(s) for the individual subject using routine procedures. In certain embodiments, pharmaceutical compositions may comprise, for example, at least about 0.1% of an active compound (e.g., a modified rAAV vector, a therapeutic agent). In other embodiments, the active compound may comprise between about 2% to about 75% of the weight of the unit, or between about 25% to about 60%, for example, and any range derivable therein.

In one aspect of methods of the present invention a heterologous nucleic acid is delivered to a cell of the vasculature or vascular tissue in vitro for purposes of administering the modified cell to a subject, e.g. through grafting or implantation of tissue. The virus particles may be introduced into the cells at the appropriate multiplicity of infection according to standard transduction methods appropriate. Titers of virus to administer can vary, depending upon the target cell type and number, and the particular virus vector, and can be determined by those of skill in the art without undue experimentation. In one embodiment, 10² infectious units, or at least about 10³ infectious units, or at least about 10⁵ infectious units are introduced to a cell.

A “therapeutically effective” amount as used herein is an amount that is sufficient to provide some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject. In certain embodiments, the therapeutically effective amount is not curative.

Administration of the virus vectors according to the present invention to a human subject or an animal in need thereof can be by any means known in the art. Preferably, the virus vector is delivered in a therapeutically effective dose in a pharmaceutically acceptable carrier. In one embodiment the vector is administered by way of a stent coated with the modified \ vector, or stent that contains the modified \ vector. A delivery sheath for delivery of vectors to the vasculature is described in U.S. patent application publication 20040193137, which is herein incorporated by reference.

Dosages of the virus vector to be administered to a subject depends upon the mode of administration, the disease or condition to be treated, the individual subject's condition, the particular therapeutic nucleic acid to be delivered, and can be determined in a routine manner. Exemplary doses for achieving therapeutic effects are delivery of virus titers of at least about 10⁵, 10⁶, 10⁷, 10⁸, 10⁹, 10¹⁰, 10¹¹, 10¹², 10¹³, 10¹⁴, 10¹⁵, transducing units or more, and any integer derivable therein, and any range derivable therein. In one embodiment, the dose for administration is about 10⁸-10¹³ transducing units. In one embodiment, the dose for administration is about 10³-10⁸ transducing units.

The dose of modified virions required to achieve a particular therapeutic effect in the units of dose in vector genomes/per kilogram of body weight (vg/kg), will vary based on several factors including, but not limited to: the route of modified virion administration, the level of nucleic acid (encoding untranslated RNA or protein) expression required to achieve a therapeutic effect, the specific disease or disorder being treated, a host immune response to the virion, a host immune response to the expression product, and the stability of the heterologous nucleic acid product. One of skill in the art can readily determine a recombinant virion dose range to treat a patient having a particular disease or disorder based on the aforementioned factors, as well as other factors that are well known in the art.

In particular embodiments, more than one administration (e.g., two, three, four or more administrations) may be employed weekly, monthly, yearly, etc.

Injectables can be prepared in conventional forms, either as liquid solutions or suspensions, solid forms suitable for solution or suspension in liquid prior to injection, or as emulsions. The vector can be delivered locally or systemically. In one embodiment the vector is administered in a depot or sustained-release formulation. Further, the virus vector can be delivered adhered to a surgically implantable matrix (e.g., as described in U.S. Patent Publication No. US-2004-0013645-A1).

The modified parvovirus vectors (e.g AAV vectors or other parvoviruses) disclosed herein may be administered by administering an aerosol suspension of respirable particles comprised of the virus vectors, which the subject inhales. The respirable particles may be liquid or solid. Aerosols of liquid particles comprising the virus vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors may likewise be produced with any solid particulate medicament aerosol generator, by techniques known in the pharmaceutical art.

In one embodiment, isolated limb perfusion, described in U.S. Pat. No. 6,177,403, and herein incorporated by reference, can also be employed to deliver the modified rAAV vector into the vasculature of an isolated limb.

In one embodiment, the modified vector of the invention is administered to vasculature tissue by inserting into the vasculature tissue a catheter in fluid communication with an inflatable balloon formed from a microporous membrane and delivering through the catheter a solution containing a vector comprising the gene of interest, see for example U.S. patent application publication 2003/0100889, which is herein incorporated by reference in its entirety.

In certain embodiments, in order to increase the effectiveness of the modified recombinant vector of the present invention, it may be desirable to combine the methods of the invention with administration of another agent, or other procedure, effective in the treatment of vascular disease or disorder. For example, in some embodiments, it is contemplated that a conventional therapy or agent including, but not limited to, a pharmacological therapeutic agent, a surgical procedure or a combination thereof, may be combined with vector administration. In a non-limiting example, a therapeutic benefit comprises reduced hypertension in a vascular tissue, or reduced restenosis following vascular or cardiovascular intervention, such as occurs during a medical or surgical procedure.

This process may involve administering the agent(s) and the vector at the same time (e.g., substantially simultaneously) or within a period of time wherein separate administration of the vector and an agent to a cell, tissue or subject produces a desired therapeutic benefit. Administration can be done with a single pharmacological formulation that includes both a modified vector and one or more agents, or by administration to the subject two or more distinct formulations, wherein one formulations includes a vector and the other includes one or more agents. In certain embodiments, the agent is an agent that reduces the immune response, e.g. a TLR-9 inhibitor, cGAS inhibitor, or rapamycin.

Administration of the modified vector may precede, be co-administered with, and/or follow the other agent(s) by intervals ranging from minutes to weeks. In embodiments where the vector and other agent(s) are applied separately to a cell, tissue or subject, one would generally ensure that a significant period of time did not expire between the time of each delivery, such that the vector and agent(s) would still be able to exert an advantageously combined effect on the cell, tissue or subject.

Administration of pharmacological therapeutic agents and methods of administration, dosages, and the like are well known to those of skill in the art (see for example, the “Physicians Desk Reference,” Goodman & Gilman's “The Pharmacological Basis of Therapeutics,” “Remington's Pharmaceutical Sciences,” and “The Merck Index, Eleventh Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

In some embodiments, the present application may be defined in any of the following paragraphs:

1. A modified recombinant AAV6 vector comprising an amino acid substitution at one or more amino acid residues selected from the group consisting of 5264, G266, N269, H272, Q457, S588 and T589 corresponding to AAV6 VP1 numbering, wherein the rAAV6 vector transduces the liver and has reduced neutralization of transduction of liver by ADK6 antibody as compared to the rAAV6 vector lacking the one or more substitutions.
2. The modified AAV6 vector of paragraph 1, further comprising a lysine (K) or arginine (R) at an amino acid site corresponding to amino acid 531 of AAV6 VP1.
3. The modified AAV6 vector of paragraph 2 comprising a K at amino acid 531.
4. The modified AAV6 vector of paragraph 2, comprising a R at amino acid 531.
5. The modified AAV6 vector of any of paragraphs 1-3, wherein at least two of the one or more amino acids are substituted.
6. The modified AAV6 vector of any of paragraphs 1-4, wherein at least three of the one or more amino acids are substituted.
7. The modified AAV6 vector of any of paragraphs 1-5, wherein at least four of the one or more amino acids are substituted.
8. The modified AAV6 vector of any of paragraphs 1-6, wherein at least five of the one or more amino acids are substituted.
9. The modified AAV6 vector of any of paragraphs 1-7, wherein at least six of the one or more amino acids are substituted.
10. The modified AAV6 vector of any of paragraphs 1-8, wherein at least seven of the one or more amino acids are substituted.
11. The modified AAV6 vector of any of paragraphs 1-9 wherein the one or more substitutions comprise conserved substitutions.
12. The modified AAV6 vector of any of paragraphs 1-9 wherein the one or more substitutions comprise non-conserved substitutions.
13. The modified rAAV6 vector of any of paragraphs 1-11, further comprising a substitution of at least one amino acids that binds to sialic acid selected form the group consisting of: N447, S472, V473, N500, T502, and W503 corresponding to AAV6 VP1 numbering.
14. The modified rAAV6 vector of any of paragraphs 1-12, further comprising one or more modified regions of amino acids selected from the group consisting of: 262-272; 382-386, 445-457, 459, 469-473, 488-489, 494-496, 499-515, 571-579, 584-589, and 593-595, corresponding to AAV6 VP1 numbering, and wherein at least one or more amino acids in the one or more modified regions are substituted.
15. A modified recombinant AAV6 vector comprising a substitution at one or more amino acid residues selected from the group consisting of S264, G266, N269, H272, Q457, S588 and T589 corresponding to AAV6 VP1 numbering, wherein the rAAV6 vector transduces the liver and has reduced neutralization of transduction by ADK6 antibody as compared to the rAAV6 vector lacking the one or more substitutions.
16. A modified rAAV6 vector comprising one or more modified regions of amino acids selected from the group consisting of: 262-272; 382-386, 445-457, 459, 469-473, 488-489, 494-496, 499-515, 571-579, 584-589, and 593-595 corresponding to AAV6 VP1 numbering wherein at least one or more amino acids in the one or more modified regions are substituted, and wherein the rAAV6 vector transduces the liver and has reduced neutralization of transduction of liver by ADK6 antibody as compared to the rAAV6 vector lacking the one or more substitutions.
17. The modified rAAV6 vector of paragraph 15 comprising K531 corresponding to AAV6 VP1 numbering.
18. The modified rAAV6 vector of paragraph 15 comprising R531 corresponding to AAV6 VP1 numbering.
19. The modified rAAV6 vector of any of paragraphs 1-17 further comprising amino acid substitution at one or more amino acid regions selected from the group consisting of: 456-459; 492-499; and 588-597.
20. The modified rAAV6 vector of any of paragraphs 1-18, comprising one or more of the amino acid sequences selected from the group consisting of: SEER at 456-499 (SEQ ID NO: 2), TPGGNATR (SEQ ID NO: 3) at 492-499; DLDPKATEVE (SEQ ID NO: 4) at 588-597.
21. The modified rAAV6 vector of any of paragraphs 1-19, wherein the vector has reduced neutralization of liver transduction by human anti-sera to unmodified rAAV6 virus as compared to neutralization of the unmodified rAAV6 vector.
22. The modified rAAV6 vector of any of paragraphs 1-19, wherein the vector has reduced neutralization of liver transduction as measured by mouse anti-sera to unmodified rAAV6 virus as compared to neutralization of the unmodified rAAV6 vector.
23. The modified rAAV6 vector of any of paragraphs 1-19, wherein the vector has reduced neutralization of liver transduction as measured by rhesus macaques anti-sera to unmodified rAAV6 virus as compared to neutralization of the unmodified rAAV6 vector.
24. A method for identifying an AAV6 virion that retains liver tropism and exhibits reduced neutralization by ADK6 antibody comprising:
a. generating a saturation mutagenesis AAV6 library wherein each of the amino acids selected from the group consisting of 5264, G266, N269, H272, Q457, 5588 and T589, are substituted with each of the 20 different natural or unnatural amino acid and in any combination of all or fewer than all positions, and wherein the AAV6 comprises either K531 or R531; and
b. performing rounds of evolution by infecting liver cells (e.g. hepatocytes) or tissue with the rAAV6 library; and
c. screening for reduced neutralization by of at least 10% ADK6 or anti-sera as compared to the corresponding unmodified AAV6 virion.
25. The method of paragraph 23, further comprising screening for the loss of sialic acid binding.
26. The method of paragraph 23, further comprising screening for the presence of sialic acid binding.
27. A method for identifying an AAV6 virion that retains liver tropism and exhibits reduced neutralization by ADK6 antibody comprising:
a. generating a saturation mutagenesis library of one or more modified regions of amino acids selected from the group consisting of: 262-272; 382-386, 445-457, 459, 469-473, 488-489, 494-496, 499-515, 571-579, 584-589, and 593-595, wherein the one or more regions are substituted with each of the 20 different natural or unnatural amino acid and in any combination of all or fewer than all positions, and wherein the AAV6 comprises either K531 or R531; and
b. performing rounds of evolution by infecting liver cells (e.g. hepatocytes) or tissue with the rAAV6 library; and
c. screening for reduced neutralization of at least 10% by ADK6 or anti-sera as compared to the corresponding unmodified AAV6 virion.
28. The method of paragraph 27, further comprising screening for the loss of sialic acid binding.
29. The method of paragraph 27, further comprising screening for the presence of sialic acid binding.
30. A modified rAAV6 vector obtained by the method of any of paragraphs 24-27, wherein the modified rAAV6 vector comprises reduced neutralization by ADK6 antibody and transduces the liver.
31. The modified rAAV6 vector of paragraph 28, wherein the vector has at least 10% reduced neutralization of liver transduction by human anti-sera to unmodified rAAV6 virus, as compared to the neutralization of unmodified rAAV6 vector.
32. The modified rAAV6 vector of paragraph 28, wherein the vector has at least 10% reduced neutralization of liver transduction by mouse anti-sera to unmodified rAAV6 virus, as compared to the neutralization of unmodified rAAV6 vector.
33. The modified rAAV6 vector of paragraph 28, wherein the vector has at least 10% reduced neutralization of liver transduction by rhesus macaques anti-sera to unmodified rAAV6 virus, as compared to neutralization of the unmodified rAAV6 vector.

All references cited herein throughout this specification, e.g. within the tables and Examples of this specification are hereby incorporated by reference in their entirety.

The examples presented below are provided as a further guide to the practitioner of ordinary skill in the art, and are not to be construed as limiting the invention in any way.

### EXAMPLES

Adeno-associated viruses (AAVs) are being developed as vectors for the treatment of genetic disorders. However, pre-existing antibodies present a significant limitation to achieving optimal efficacy for the AAV gene delivery system. Efforts aimed at engineering vectors with the ability to evade the immune response include identification of residues on the virus capsid important for these interactions and changing them. Here K531 is identified as the determinant of monoclonal antibody ADK6 recognition by AAV6, and not the closely related AAV1. The AAV6 ADK6 complex structure was determined by cryo-electron microscopy and the footprint confirmed by cell-based assays. The ADK6 footprint overlaps previously identified AAV antigenic regions and neutralizes by blocking essential cell surface glycan attachment sites. This study thus expands the available repertoire of AAV-antibody information that can guide the design of host immune escaping AAV vectors able to maintain capsid functionality.

### Example 1 Cryo-Reconstruction of the AAV6-ADK6 Complex

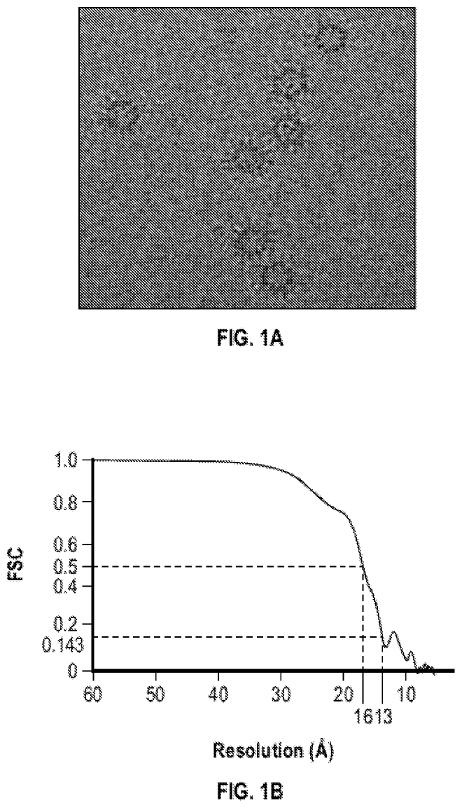

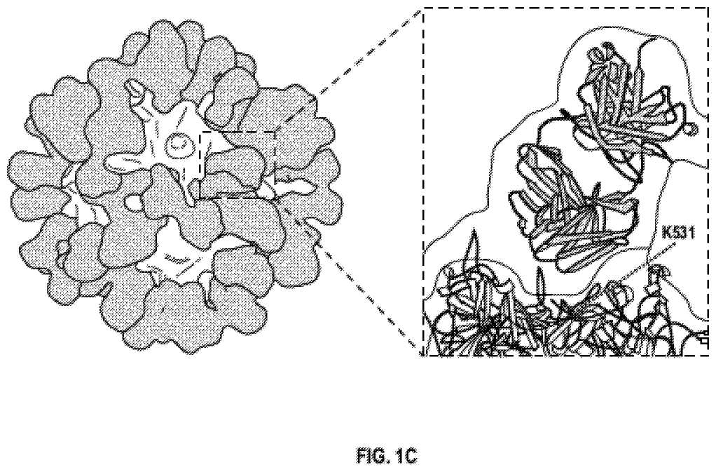

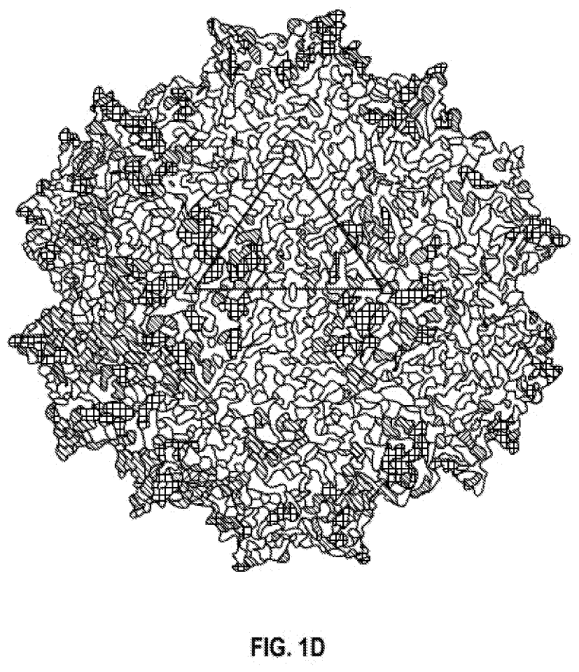

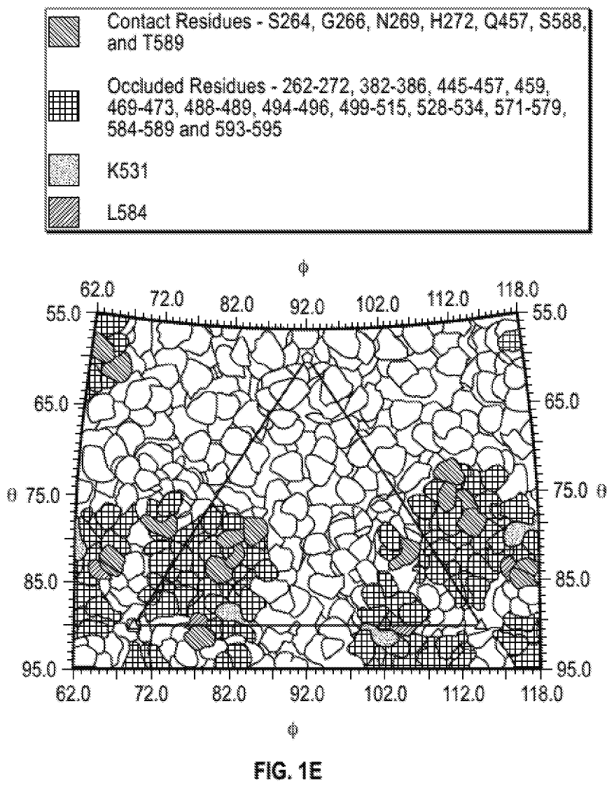

The cryo-EM reconstruction of the AAV6:ADK6 FAb complex, determined to 16 Å resolution (FSC 0.5), shows ADK6 FAbs bound to the side of the 3-fold protrusion across the 2-fold axis of the AAV6 capsid surface suggesting bivalent binding (FIG. 1A-C). Consistent with this observation, a complex with an intact ADK6 IgG (un-cleaved) showed a similar structure to the FAb alone with no ordered density for the Fc (data not shown). The ADK6 binding site on the AAV6 capsid is within a common antigenic site utilized by the other AAVs, the 3-fold protrusions and the ⅖-fold wall (Tseng et al., 2015). These epitopes are regions of high sequence and structural diversity and shown to be important for receptor recognition and transduction by the AAVs. The predicted contact residues between AAV6 and the ADK6 FAb in the pseudo-atomic model fitted into the reconstructed density map, with CC of 0.93, are shown in (FIG. 1C-E).

The AAV6 capsid surface residues which make contact with the docked ADK6 FAb model residues identified by the PDBePISA program are S264, G266, N269, H272, Q457, S588, and T589. Residues S264, G266, N269, and H272 are located on one 3-fold symmetry related monomer, Q457 on a second 3-fold related monomer, and residues S588 and T589 were located on the third 3-fold related monomer. Additionally, the ADK6 Fab footprint occludes other residues surrounding the model predicted contact residues: 262-272, 382-386, 445-457, 459, 469-473, 488-489, 494-496, 499-515, 528-534, 571-579, 584-589 and 593-595. Significantly, two of the residues that differ between AAV1 and AAV6, 531 and 584, are located in the occluded region (FIGS. 1D and E). Only AAV6, but not AAV1, binds to ADK6 (Sonntag et al., 2011). Thus, the footprint implied that the specificity of the AAV6:ADK6 interaction is dictated by 531, 584 or both.

### Example 2 Reciprocal AAV1 and AAV6 Vectors are Comparable to Wild-Type in Capsid Assembly, Genome Titer, and Transduction Efficiency

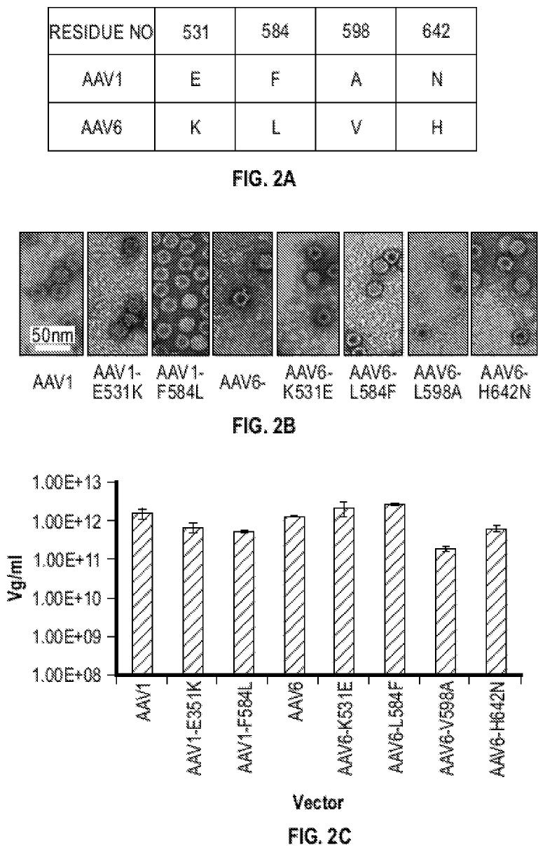

The AAV1 and AAV6 variants purified by AVB column chromatography showed assembled capsids when visualized by negative stain EM (FIG. 2B), and had packaged genome titers in the 10¹⁰-10¹³ vg/ml range, which is comparable to WT virus (FIG. 2C). The AAV6-H642N variant, with a change on the interior surface of the capsid, also assembled capsids and packaged genome at levels comparable to WT. The transduction phenotype of the WT and variants were compared in the absence of antibody. The AAV1 variants had transduction efficiencies of ˜160% compared to the WT virus while AAV6 variants were in the range ˜95 (for AAV6-V598A) to 160% (for AAV6-L584F). The transduction efficiency of AAV6-K531E was ˜120% compared to WT AAV6. These observations confirmed that the mutations made had no significant effect on capsid assembly, genome packaging or transduction efficiency.

### Example 3 AAV6 K531 is Responsible for ADK6 Recognition

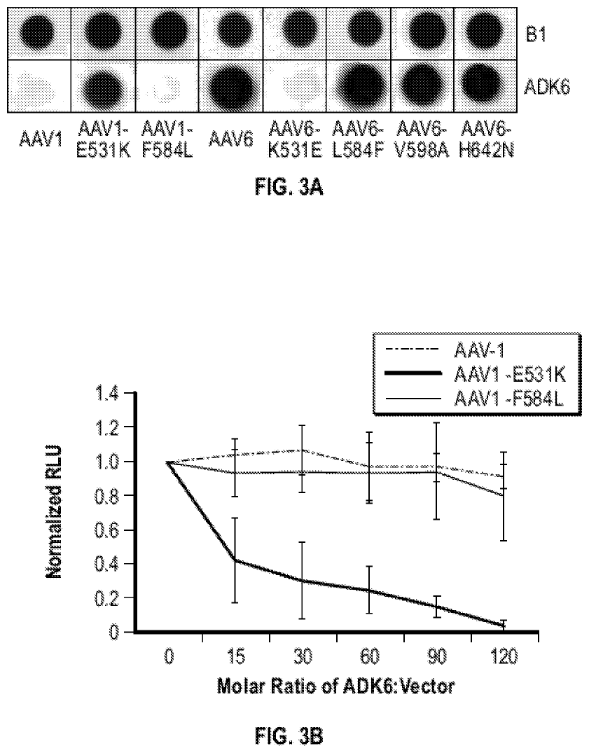

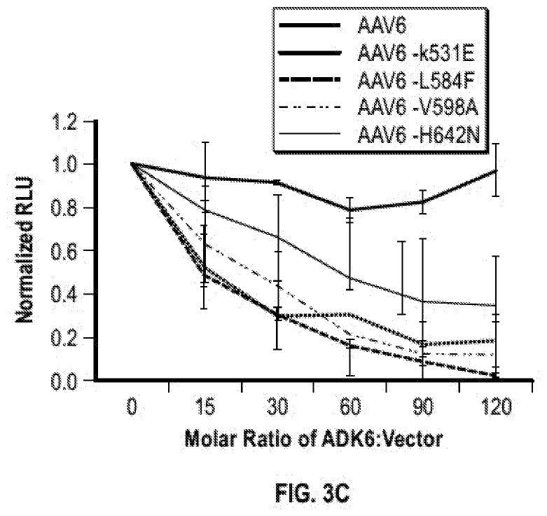

Immunoblots of AAV1 and AAV6 with ADK6 confirmed the recognition of AAV6 by ADK6 and the escape by AAV1 (FIG. 3A). Importantly, ADK6 recognizes variant AAV1-E531K, with an E to K change switching the AAV1 residue type to AAV6, but not AAV1-F584 with an L to F switch at the second occluded position. This observation identified AAV6-K531 as being the determinant of ADK6 recognition. Consistent with this conclusion, ADK6 recognizes AAV6-L584F and AAV6-H642N and not AAV6-K531E (FIG. 3A). AAV1 and AAV1-F584L escape from ADK6 while AAV1-E531K is neutralized by ADK6 at a molar ratio of ˜15 FAb molecules per capsid (FIG. 3B). This is only 25% binding site occupancy. ADK6 results in 50% inhibition of transduction for AAV6 and AAV6-L584F, also at a molar ratio of ˜15 FAb molecules per capsid, AAV6-V598A with ˜20 FAb molecules, and AAV6-H642N with ˜60 FAb molecules (FIG. 3C). On the other hand, AAV6-K531E escapes antibody recognition at up to a molar ratio of 120 FAb molecules per capsid, a saturation of 2 FAb molecules per VP binding site (FIG. 3C). These observations confirm the role of K531 as the determinant of the specificity of ADK6 for AAV6 and highlights the important contributions of capsid residues that may not make direct contact with FAb residues but are part of the footprint in virus-antibody interactions.

### Example 4 ADK6 Binding is Predicted to Sterically Hinder AAV6 Glycan Binding and has a Footprint that Overlaps Previously Defined Epitopes

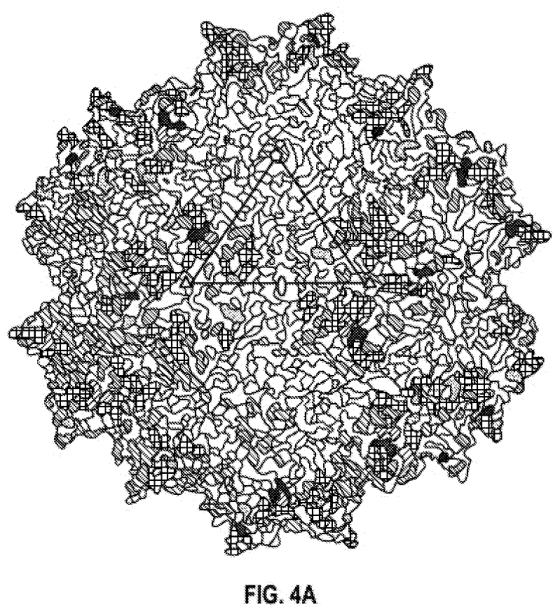

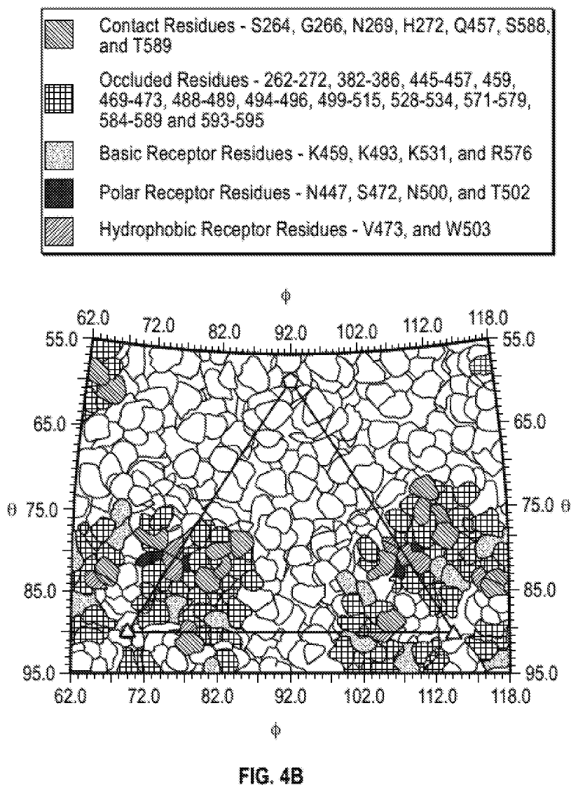

AAV6 is a dual glycan receptor binding serotype that utilizes both HS and SIA for cellular infection (Huang et al., 2016; Wu et al., 2006). The ADK6 footprint covers a large area of the AAV6 capsid surface including the previously structurally mapped SIA glycan receptor binding site in addition to K531 reported to be responsible for its HS binding (FIGS. 4A and 4B) (Huang et al., 2013; Wu et al., 2006). The ability of ADK6 to block transduction by AAV6 indicates that ADK6 neutralizes AAV6 infection at the entry step, likely by steric hindrance of both glycan interactions. The block of SIA interaction is similar to the mechanism proposed for ADK1a neutralization of AAV1 and AAV6 which shares regions of the ADK6 footprint at the top of the 3-fold region (Huang et al., 2016; Tseng and Agbandje-McKenna, 2014). This mechanism is different to those reported for the A20 neutralization of AAV2 and ADK8 neutralization of AAV8 (Gurda et al., 2012; Huttner et al., 2003; McCraw et al., 2012; Tseng et al., 2015). These antibodies are proposed to neutralize at a post-entry step, with A20 acting in the nucleus and ADK8 blocking nuclear entry and resulting in perinuclear accumulation. Importantly, while an AAV8 cell surface glycan receptor is unknown, AAV2 binds its HS receptor at the 3-fold axis, a region disparate from the A20 footprint located at the 2/−5-fold wall (McCraw et al., 2012).

The ADK6 footprint on AAV6 overlaps that previously mapped for other AAV-antibody interactions, including AAV8-ADK8, AAV1-ADK1a, and regions of AAV1-5H7, AAV6-5H7, and AAV2-A20 on the 3-fold protrusions and the 2/5-fold walls (Gurda et al., 2012; Tseng and Agbandje-McKenna, 2014; Tseng et al., 2015; Tseng et al., 2016). This structure thus adds to the library of antigenic footprint information being accumulated for the AAVs and will inform the engineering of a generation of AAV vectors with the ability to evade pre-existing host immune responses during the vector re-administration.

This study, using a combination of structure, site directed mutagenesis, and cell binding assays, identified a single residue, K531, as conferring antigenic selectivity between the closely related AAV1 and AAV6. Significantly, repeat administration of the approved AAV1 based lipoprotein lipase gene vector treatment will require the use of an antigenic variant with similar transduction properties. The observation that position 531 of AAV1/6 is able to provide immune escape properties is information that can inform AAV1 engineering for future use in these patients as well as the development of additional AAV1 and AAV6 vectors.

Methods

Production and Purification of AAV6 Virus-Like Particles.

The production and purification of AAV6 virus-like particles (VLPs) using the Baculovirus/SF9 expression system has been previously described (DiMattia et al., 2005; Miller et al., 2006; Ng et al., 2010). Briefly, a baculovirus packaging a gene containing the DNA for expressing the AAV6 VP2 and VP3 was made using the Bac-to-Bac system according to the manufacturer's instructions (Invitrogen) and used to infect SF9 cells. The cell pellet from the infection was resuspended in TNTM buffer (25 mM Tris-HCl, pH 8.0, 100 mM NaCl, 0.2% Triton X-100, 2 mM MgCl2), freeze/thawed 3× with enzonase (Promega) treatment at 37° C. after the second thaw, and clarified by centrifugation at 10,000 rpm in a JA-20 rotor at 4° C. for 20 min. The supernatant was loaded on a 20% sucrose cushion (w/v of sucrose in TNTM buffer) and the sample centrifuged at 45,000 rpm on a Ti70 rotor at 4° C. for 3 hr. The supernatant was discarded and the pellet was resuspended in TNTM and left stirring overnight at 4° C. The resuspended pellet was loaded onto a 5-40% (w/v) sucrose step gradient and the sample centrifuged at 35,000 rpm in an SW41 rotor for at 4° C. for 3 hr. The VLP containing fraction were collected by fractionation, dialyzed into Buffer A (20 mM Tris-HCl, pH 8.5, 15 mM NaCl) and the sample further purified by ion exchange chromatography before use.

A 1 ml anion exchange (Q column, GE Healthcare) was equilibrated with Buffer A and Buffer B (20 mM Tris-HCl, pH 8.5, 500 mM NaCl). The sample was loaded on the column at a flow rate of 0.5 ml/min, the column was washed with 10 column volume (CV) of Buffer A, and the sample was eluted with a 5 CV gradient from 0-100% Buffer B (Zolotukhin et al., 2002). Five hundred microliter fractions were collected and screened to identify the fractions containing AAV6 VP. These fractions were pooled, buffer exchanged into PBS, concentrated to 1 mg/ml, and analyzed by SDS PAGE and negative stain electron microscopy (EM) to check for purify and capsid integrity, respectively.

Purification of ADK6 IgG Antibodies.

The ADK6 immunoglobulin G (IgG) antibody was produced by the University of Florida Hybridoma Core Lab as previously described (Kuck et al., 2007; Tseng et al., 2016). The ADK6 hybridoma supernatant was diluted 1:5 with PBS and loaded onto a 1 ml HiTrap protein G HP column (GE Healthcare), washed with 10 CV of PBS, eluted with 0.5 ml of Glycine-HCl at pH 2.7, and neutralized with 50 μl of neutralization buffer (1 M Tris-HCl pH 10). The purified IgG was buffer exchanged into 20 mM Sodium Phosphate pH 7.0, 10 mM EDTA, and concentrated for papain cleavage.

ADK6 Fragment Antibody (FAb) Generation and Purification.

Cysteine HCl was added to the papain digestion buffer (20 mM sodium phosphate pH 7.0, 10 mM EDTA) immediately prior to use. Concentrated IgGs were incubated with immobilized papain (Pierce) at an enzyme:substrate ratio of 1:160 at 37° C. for 16 h. An equal volume of papain stop buffer (10 mM Tris-HCl pH 7.5) was added to stop the cleavage process and the mixture was centrifuged at 1500×g for 2 min to separate the sample from the immobilized papain. The FAbs were separated from the undigested IgG and fragment crystallizable (Fc) fragments on a HiTrap Protein A column (GE Healthcare).

The FAbs were collected in the wash and flowthrough fractions, buffer exchanged into PBS, and concentrated for use.

AAV6-ADK6 FAb Complex Formation.

AAV6 VLPs at a concentration of 1 mg/ml and ADK6 FAbs at a concentration of 1 mg/ml were mixed at a molar ratio of 1:1 and 2:1 FAb:VP binding site, and the mixture was incubated at 4° C. for 1 h. The complexes were examined by negative stain EM on a Spirit microscope (FEI) to confirm capsid decoration by FAbs prior to vitrification for cryo-EM data collection.

AAV6-ADK6 FAb Complex Cryo-EM Data Collection.

Three microliters of the AAV6-ADK6 Fab complex mixture were loaded onto C-Flat holey carbon grids (CF-2/2-4C-50, Protochips Inc.) that were glow discharged for 1 min prior to use, and vitrified by plunge freezing into liquefied ethane in a Vitrobot Mark IV (FEI). The frozen grids were transferred to liquid nitrogen and then into a FEI Technai TF20 transmission electron microscope operating at 200 kV. Cryo micrographs were collected using a defocus range of 2.5-3.0 μm and total dosage of 20e-/Å2 per image. Thirty-six micrographs were collected with a Gatan Ultra Scan 4000 CCD camera at a step size of 1.82 Å/pixel.

Cryo-EM and Image Reconstruction of the AAV6-ADK6 FAb Complex.

The RobEM software package (available on the world wide web at cryoEM.ucsd.edu/programs.shtm) was used to extract decorated AAV6 VLPs (complexed) particles from each micrograph. The defocus level for each micrograph was estimated using the CTFFIND3 application (Mindell and Grigorieff, 2003) incorporated into the AUTO3DEM application (Yan et al., 2007a; Yan et al., 2007b). Preprocessing of the selected particles to remove blemishes, correct linear gradient, normalize, and apodize the images used the “autopp” subroutine within the AUTO3DEM software package and an initial model, at ˜25 Å resolution, was generated for searching and initiating the refinement of each particle origin and orientation using the same application (Yan et al., 2007a). Following an initial 10 cycles of search and refinement, the data set was “re-boxed” and “re-centered” using the refined particle center and orientation information, and the images were corrected to compensate for the effect of phase reversal in the contrast transfer function (CTF) followed by additional cycles of refinement also within AUTO3DEM (Yan et al., 2007b). The final resolution was determined by the Fourier shell correlation (FSC) threshold of 0.5 (van Heel and Schatz, 2005). The images of the reconstructed map were illustrated using the Chimera software package (Pettersen et al., 2004).

Pseudo-Atomic Model Fitting and Identification of the ADK6 Antibody Footprint.

The AAV6 60 mer VP3 capsid coordinates were generated by icosahedral matrix multiplication using the Oligomer generator subroutine using the Viperdb online server (available on the world wide web at viperdb.scripps.edu/) (Carrillo-Tripp et al., 2009) from the AAV6 crystal structure (RCSB PDB ID no: 3OAH). The coordinates were fitted into the cryo-EM reconstructed complex density map by rigid body rotations and translations using the Chimera program (Pettersen et al., 2004). This 60 mer docked with a correlation coefficient (CC) of 0.94. To enable model building into the FAb density, a difference map, subtracting a scaled density map generated for the docked 60 mer model from the AAV6-ADK6 complex map, was generated. A generic FAb structure (PDB ID no: 2FBJ) was fitted into the resulting positive difference density map representing the FAb interacting with the reference AAV6 VP3 monomer (chain A) using the Chimera program (Pettersen et al., 2004). The coordinates for the reference monomer was extracted from the docked 60 mer and together with the docked Fab model was used to generate a 60 mer using Viperdb (available on the world wide web at viperdb.scripps.edu/) (Carrillo-Tripp et al., 2009). This complex 60 mer was then re-docked into the reconstructed complex density map and the CC was similar at 0.93. To determine the interacting residues between the AAV6 capsid and ADK6 FAbs the PDBePISA (available on the world wide web at ebi.ac.uk/msd-srv/prot_int/) (Krissinel and Henrick, 2007) and COOT (Emsley et al., 2010) software packages were used. Images for the co-ordinates of the fitted complex were generated using the PyMol program (available on the world wide web at pymol.org/) (Schrödinger, 2017).

Recombinant Wild-Type and Variant AAV1 and AAV6 Vector Production and Purification.

To identify the critical residue determinant for ADK6 recognition by AAV6 (and not AAV1) recombinant wild-type (WT) AAV1 (rAAV1) and AAV6 (rAAV6), and reciprocal single site-directed variants, at the equivalent capsid surface amino acid positions 531 and 584, K and F in AAV6 and E and L in AAV1, respectively, located within the ADK6 footprint, and AAV6-V598A were made for testing by native immunoblot and infectivity in the presence of ADK6. As a negative control of a differing non-footprint residue (interior capsid surface) and control for AAV6 capsid assembly in the presence of the amino acid substitution, the reciprocal AAV6-H642N variant was also created for testing. These variants were made as previously described in the pXR1 and pXR6 backgrounds, for AAV1 and AAV6, respectively (Wu et al., 2006).

To produce WT and variant rAAV1 and rAAV6 vectors, monolayers of HEK293 cells, at 70% confluency, were triply transfected with 18 μg of WT and mutant pXRAAV1 and pXRAAV6 plasmids, 18 μg of pTR-UF3-Luciferase (luciferase gene between AAV2 inverted terminal repeats), and 54 μg of the helper plasmid pXX6, with 190 μl of Polyethyleneimine (1 mg/ml) per 15 cm plate. Ten 15 cm plates were transfected for each vector followed by incubation at 37° C. for 72 h in the presence of 5% CO2. Post transfection, the cells were harvested and centrifuged at 1100×g for 15 min. The supernatant for each vector was precipitated with 10% PEG 8000 (Fisher) and the cell pellets were resuspended in 10 ml TD buffer (1×PBS, 5 mM MgCl2 and 2.5 mM KCl, pH7.4) and freeze/thawed 3× to release virus from the cells. Both the PEG precipitated virus resuspended in TD buffer and the resuspended cell lysate were Benzonase (Novagen) treated at 37° C. for 1 h followed by clarification by centrifugation at 10,000 rpm in a JA-20 rotor at 4° C. for 20 min. The genome containing vectors were separated from the empty capsids by a step Iodixanol radient (Zolotukhin et al., 2002). In brief, the clarified supernatants were loaded onto a 15-60% step iodixanol gradient. The 40/60% interface or vector containing fractions was collected and diluted with 10×TD buffer. The genome containing vectors were further purified by AVB (Thermo Fisher) affinity column chromatography (Mietzsch et al., 2014). A 1 ml AVB column was equilibrated with 10 ml TD buffer, and the diluted vector containing fractions were loaded at 1 ml/min. The purified vector was eluted with 10 ml or 10 CV elution buffer (0.1M Na Acetate pH=2.5 and 0.75M NaCl at 0.5 ml fractions). The elution fractions were diluted with 50 μl neutralization buffer (1M Tris-HCl pH=10). The purified vectors were buffer exchanged into PBS buffer and quantified by UV spectrometry analysis and qPCR.

Native Immunoblots.

To confirm the interaction of AAV6 with ADK6 and the lack of recognition of AAV1 by the antibody, and to further delineate the specific residue(s) important for the capsid antibody binding, the purified rAAV1 and rAAV6 vectors and the single residue variants, rAAV1-E531K, rAAV1-F584L, rAAV6-K531E, rAAV6-L584F, rAAV6-H642N vectors (FIG. 2A) were probed by native immunoblot with ADK6. One hundred ng of purified intact vectors were loaded on a nitrocellulose membrane. The membrane was blocked with 5% milk (w/v) in PBS and 0.05% Tween (1 h), followed by 1 h incubation with ADK6 (0.5 mg/ml) diluted 1:3000. The rAAV-ADK6 complexes were probed with the Horse Radish Peroxidase (HRP) anti-mouse secondary antibody diluted 1:5000. The membrane was finally probed with chemiluminescent and detected on a Kodak film. The film image was documented with a Gel Doc (Biorad). As a positive control 100 ng of rAAV vectors were boiled at 100° C. for 5 min and the sample loaded on a nitrocellulose membrane. The sample was then probed with the B1 antibody that recognizes the C-terminus of the rAAV1 and rAAV6 VP. This C-terminus epitope is only available when the capsid is denatured (Wobus et al., 2000).

Neutralization Assays.

To determine if binding of ADK6 to the WT and variant rAAV vectors is neutralizing in vitro, purified vectors were used to infect HEK293T cells in the presence of the antibody as previously described (Tseng et al., 2015). Briefly, HEK293T cells were seeded in 96 well plates at 1.25×104 cells/well for 24 h to reach 70% confluency. The purified WT and mutant vectors (2.5×109 viral genome (vg)) were incubated at virus particle: ADK6 IgG molecular ratios of 1:0, 1:15, 1:30, 1:60, 1:90 and 1:120 in PBS in a final volume of 30 μl in unsupplemented DMEM (Gibco) at 37° C. for 1 h. After this incubation period, the media was aspirated from the cells, the complex sample was added to 70 μl of DMEM supplemented with 10% FBS and 1% Antibiotic and antimytotic (ABAM), and the mixture was added to the cells. The cells were incubated at 37° C. for 48 h in the presence of 5% CO2. The cells were harvested, washed with PBS, lysed, and transduction level determined by the Luciferase Assay System (Promega)according to the manufacturer's instruction.

### References for Examples 1-4

Agbandje-McKenna, M., Chapman, M. S., 2006. Structure function relationships, in: M. E. Bloom, S. F. C., R. M. Linden, C. R. Parrish, and J. R. Kerr (Ed.), In Parvoviruses. Edward Arnold, &#x9; Ltd, London, pp. 125-139.
Boutin, S., Monteilhet, V., Veron, P., Leborgne, C., Benveniste, O., Montus, M. F., Masurier, C., 2010.
Prevalence of serum IgG and neutralizing factors against adeno-associated virus (AAV) types 1, 2, 5, 6, 8, and 9 in the healthy population: implications for gene therapy using AAV vectors. Hum Gene Ther 21, 704-712.
Buller, R. M., Rose, J. A., 1978. Characterization of adenovirus-associated virus-induced polypeptides in KB cells. J Virol 25, 331-338.
Buning, H., Braun-Falco, M., Hallek, M., 2004. Progress in the use of adeno-associated viral vectors for gene therapy. Cells Tissues Organs 177, 139-150.
Carrillo-Tripp, M., Shepherd, C. M., Borelli, I. A., Venkataraman, S., Lander, G., Natarajan, P., Johnson, J. E., Brooks, C. L., 3rd, Reddy, V. S., 2009. VIPERdb2: an enhanced and web API enabled relational database for structural virology. Nucleic Acids Res 37, D436-442.
Chapman, M. S., Agbandje-McKenna, M., 2006. Atomic structure of viral particles, in: M. E. Bloom, S. F. C., R. M. Linden, C. R. Parrish, and J. R. Kerr (Ed.), In Parvoviruses. Edward Arnold, &#x9; Ltd, London, pp. 109-123.
DiMattia, M., Govindasamy, L., Levy, H. C., Gurda-Whitaker, B., Kalina, A., Kohlbrenner, E., Chiorini, J. A., McKenna, R., Muzyczka, N., Zolotukhin, S., Agbandje-McKenna, M., 2005. Production, purification, crystallization and preliminary X-ray structural studies of adeno-associated virus serotype 5. Acta Crystallogr Sect F Struct Biol Cryst Commun 61, 917-921. Emsley, P., Lohkamp, B., Scott, W. G., Cowtan, K., 2010. Features and development of Coot. Acta Crystallogr D Biol Crystallogr 66, 486-501.
Gao, G., Vandenberghe, L. H., Alvira, M. R., Lu, Y., Calcedo, R., Zhou, X., Wilson, J. M., 2004. Clades of Adeno-associated viruses are widely disseminated in human tissues. J Virol 78, 6381-6388.
Gao, G. P., Alvira, M. R., Wang, L., Calcedo, R., Johnston, J., Wilson, J. M., 2002. Novel adeno-associated viruses from rhesus monkeys as vectors for human gene therapy. Proc Natl Acad Sci USA 99, 11854-11859.
Gaudet, D., Methot, J., Kastelein, J., 2012. Gene therapy for lipoprotein lipase deficiency. Curr Opin Lipidol 23, 310-320.
Gurda, B. L., Raupp, C., Popa-Wagner, R., Naumer, M., Olson, N. H., Ng, R., McKenna, R., Baker, T. S., Kleinschmidt, J. A., Agbandje-McKenna, M., 2012. Mapping a neutralizing epitope onto the capsid of adeno-associated virus serotype 8. J Virol 86, 7739-7751.
Huang, L. Y., Patel, A., Ng, R., Miller, E. B., Halder, S., McKenna, R., Asokan, A., Agbandje-McKenna, M., 2016. Characterization of the Adeno-Associated Virus 1 and 6 Sialic Acid Binding Site. J Virol 90, 5219-5230.
Huang, X., Hartley, A. V., Yin, Y., Herskowitz, J. H., Lah, J. J., Ressler, K. J., 2013. AAV2 production with optimized N/P ratio and PEI-mediated transfection results in low toxicity and high titer for in vitro and in vivo applications. J Virol Methods 193, 270-277.
Hurlbut, G. D., Ziegler, R. J., Nietupski, J. B., Foley, J. W., Woodworth, L. A., Meyers, E., Bercury, S. D., Pande, N. N., Souza, D. W., Bree, M. P., Lukason, M. J., Marshall, J., Cheng, S. H., Scheule, R. K., 2010. Preexisting immunity and low expression in primates highlight translational challenges for liver-directed AAV8-mediated gene therapy. Mol Ther 18, 1983-1994.
Huttner, N. A., Girod, A., Perabo, L., Edbauer, D., Kleinschmidt, J. A., Buning, H., Hallek, M., 2003. Genetic modifications of the adeno-associated virus type 2 capsid reduce the affinity and the neutralizing effects of human serum antibodies. Gene Ther 10, 2139-2147.Krissinel, E., Henrick, K., 2007. Inference of macromolecular assemblies from crystalline state. J Mol Biol 372, 774-797.
Kuck, D., Kern, A., Kleinschmidt, J. A., 2007. Development of AAV serotype-specific ELISAs using novel monoclonal antibodies. J Virol Methods 140, 17-24.
Li, C., Narkbunnam, N., Samulski, R. J., Asokan, A., Hu, G., Jacobson, L. J., Manco-Johnson, M. J., Monahan, P. E., 2012. Neutralizing antibodies against adeno-associated virus examined prospectively in pediatric patients with hemophilia. Gene Ther 19, 288-294.
Limberis, M. P., Vandenberghe, L. H., Zhang, L., Pickles, R. J., Wilson, J. M., 2009. Transduction efficiencies of novel AAV vectors in mouse airway epithelium in vivo and human ciliated airway epithelium in vitro. Mol Ther 17, 294-301.
Manno, C. S., Pierce, G. F., Arruda, V. R., Glader, B., Ragni, M., Rasko, J. J., Ozelo, M. C., Hoots, K., Blatt, P., Konkle, B., Dake, M., Kaye, R., Razavi, M., Zajko, A., Zehnder, J., Rustagi, P. K.,
Nakai, H., Chew, A., Leonard, D., Wright, J. F., Lessard, R. R., Sommer, J. M., Tigges, M., Sabatino, D., Luk, A., Jiang, H., Mingozzi, F., Couto, L., Ertl, H. C., High, K. A., Kay, M. A., 2006. Successful transduction of liver in hemophilia by AAV-Factor IX and limitations imposed by the host immune response. Nat Med 12, 342-347.
McCraw, D. M., O'Donnell, J. K., Taylor, K. A., Stagg, S. M., Chapman, M. S., 2012. Structure of adeno-associated virus-2 in complex with neutralizing monoclonal antibody A20. Virology 431, 40-49.
Mendell, J. R., Rodino-Klapac, L. R., Rosales-Quintero, X., Kota, J., Coley, B. D., Galloway, G., Craenen, J. M., Lewis, S., Malik, V., Shilling, C., Byrne, B. J., Conlon, T., Campbell, K. J., Bremer, W. G., Viollet, L., Walker, C. M., Sahenk, Z., Clark, K. R., 2009. Limb-girdle muscular dystrophy type 2D gene therapy restores alpha-sarcoglycan and associated proteins. Ann Neurol 66, 290-297.
Mietzsch, M., Grasse, S., Zurawski, C., Weger, S., Bennett, A., Agbandje-McKenna, M., Muzyczka, N., Zolotukhin, S., Heilbronn, R., 2014. OneBac: platform for scalable and high-titer production of adeno-associated virus serotype 1-12 vectors for gene therapy. Hum Gene Ther 25, 212-222.
Miller, E. B., Gurda-Whitaker, B., Govindasamy, L., McKenna, R., Zolotukhin, S., Muzyczka, N., Agbandje-McKenna, M., 2006. Production, purification and preliminary X-ray crystallographic studies of adeno-associated virus serotype 1. Acta Crystallogr Sect F Struct Biol Cryst Commun 62, 1271-1274.
Mindell, J. A., Grigorieff, N., 2003. Accurate determination of local defocus and specimen tilt in electron microscopy. J Struct Biol 142, 334-347.
Mori, S., Takeuchi, T., Enomoto, Y., Kondo, K., Sato, K., Ono, F., Sata, T., Kanda, T., 2008. Tissue distribution of cynomolgus adeno-associated viruses AAV10, AAV11, and AAVcy. 7 in naturally infected monkeys. Arch Virol 153, 375-380.
Nathwani, A. C., Tuddenham, E. G., Rangaraj an, S., Rosales, C., McIntosh, J., Linch, D. C., Chowdary, P., Riddell, A., Pie, A. J., Harrington, C., O'Beirne, J., Smith, K., Pasi, J., Glader, B., Rustagi, P., Ng, C. Y., Kay, M. A., Zhou, J., Spence, Y., Morton, C. L., Allay, J., Coleman, J., Sleep, S., Cunningham, J. M., Srivastava, D., Basner-Tschakarj an, E., Mingozzi, F., High, K. A., Gray, J. T., Reiss, U. M., Nienhuis, A. W., Davidoff, A. M., 2011. Adenovirus-associated virus vector-mediated gene transfer in hemophilia B. N Engl J Med 365, 2357-2365.
Ng, R., Govindasamy, L., Gurda, B. L., McKenna, R., Kozyreva, 0.G., Samulski, R. J., Parent, K. N., Baker, T. S., Agbandje-McKenna, M., 2010. Structural characterization of the dual glycan binding adeno-associated virus serotype 6. J Virol 84, 12945-12957.
Pettersen, E. F., Goddard, T. D., Huang, C. C., Couch, G. S., Greenblatt, D. M., Meng, E. C., Ferrin, T. E., 2004. CSF Chimera--a visualization system for exploratory research and analysis. J Comput Chem 25, 1605-1612.
Rose, J. A., Maizel, J. V., Jr., Inman, J. K., Shatkin, A. J., 1971. Structural proteins of adenovirus-associated viruses. J Virol 8, 766-770.
Schmidt, M., Voutetakis, A., Afione, S., Zheng, C., Mandikian, D., Chiorini, J. A., 2008. Adeno-associated virus type 12 (AAV12): a novel AAV serotype with sialic acid- and heparan sulfate proteoglycan-independent transduction activity. J Virol 82, 1399-1406.
Schrödinger, L., 2017. The PyMOL Molecular Graphics System, Version 2.0. Snijder, J., van de Waterbeemd, M., Damoc, E., Denisov, E., Grinfeld, D., Bennett, A., Agbandje-McKenna, M., Makarov, A., Heck, A. J., 2014. Defining the stoichiometry and cargo load of viral and bacterial nanoparticles by Orbitrap mass spectrometry. J Am Chem Soc 136, 7295-7299.
Sonntag, F., Kother, K., Schmidt, K., Weghofer, M., Raupp, C., Nieto, K., Kuck, A., Gerlach, B., Bottcher, B., Muller, O. J., Lux, K., Horer, M., Kleinschmidt, J. A., 2011. The assembly-activating protein promotes capsid assembly of different adeno-associated virus serotypes. J Virol 85, 12686-12697.
Tseng, Y. S., Agbandje-McKenna, M., 2014. Mapping the AAV Capsid Host Antibody Response toward the Development of Second Generation Gene Delivery Vectors. Front Immunol 5, 9.
Tseng, Y. S., Gurda, B. L., Chipman, P., McKenna, R., Afione, S., Chiorini, J. A., Muzyczka, N., Olson, N. H., Baker, T. S., Kleinschmidt, J., Agbandje-McKenna, M., 2015. Adeno-associated virus serotype 1 (AAV1)- and AAVS-antibody complex structures reveal evolutionary commonalities in parvovirus antigenic reactivity. J Virol 89, 1794-1808.
Tseng, Y. S., Vliet, K. V., Rao, L., McKenna, R., Byrne, B. J., Asokan, A., Agbandje-McKenna, M., 2016. Generation and characterization of anti-Adeno-associated virus serotype 8 (AAV8) and anti-AAV9 monoclonal antibodies. J Virol Methods 236, 105-110.
van Heel, M., Schatz, M., 2005. Fourier shell correlation threshold criteria. J Struct Biol 151, 250-262.
Wagner, J. A., Messner, A. H., Moran, M. L., Daifuku, R., Kouyama, K., Desch, J. K., Manley, S., Norbash, A. M., Conrad, C. K., Friborg, S., Reynolds, T., Guggino, W. B., Moss, R. B., Carter, B. J., Wine, J. J., Flotte, T. R., Gardner, P., 1999. Safety and biological efficacy of an adeno-associated virus vector-cystic fibrosis transmembrane regulator (AAV-CFTR) in the cystic fibrosis maxillary sinus. Laryngoscope 109, 266-274.
Wobus, C. E., Hugle-Dorr, B., Girod, A., Petersen, G., Hallek, M., Kleinschmidt, J. A., 2000. Monoclonal antibodies against the adeno-associated virus type 2 (AAV-2) capsid: epitope mapping and identification of capsid domains involved in AAV-2-cell interaction and neutralization of AAV-2 infection. J Virol 74, 9281-9293.
Wu, Z., Asokan, A., Grieger, J. C., Govindasamy, L., Agbandje-McKenna, M., Samulski, R. J., 2006. Single amino acid changes can influence titer, heparin binding, and tissue tropism in different adeno-associated virus serotypes. J Virol 80, 11393-11397.
Xiao, C., Rossmann, M. G., 2007. Interpretation of electron density with stereographic roadmap projections. J Struct Biol 158, 182-187.
Yan, X., Dryden, K. A., Tang, J., Baker, T. S., 2007a. Ab initio random model method facilitates 3D reconstruction of icosahedral particles. J Struct Biol 157, 211-225.
Yan, X., Sinkovits, R. S., Baker, T. S., 2007b. AUTO3DEM—an automated and high throughput program for image reconstruction of icosahedral particles. J Struct Biol 157, 73-82.
Zadori, Z., Szelei, J., Lacoste, M. C., Li, Y., Gariepy, S., Raymond, P., Allaire, M., Nabi, I. R., Tijssen, P., 2001. A viral phospholipase A2 is required for parvovirus infectivity. Dev Cell 1, 291-302.
Zolotukhin, S., Potter, M., Zolotukhin, I., Sakai, Y., Loiler, S., Fraites, T. J., Chiodo, V. A., Phillipsberg, T., Muzyczka, N., Hauswirth, W. W., Flotte, T. R., Byrne, B. J., Snyder, R. O., 2002. Production and purification of serotype 1, 2, and 5 recombinant adeno-associated viral vectors. Methods 28, 158-167.

Example 5 Saturation mutagenesis generation of AAV capsid libraries for additional modified rAAV6 vectors that evade neutralization and retain liver tropism.

AAV libraries will be engineered through saturation mutagenesis of amino acid residues S264, G266, N269, H272, Q457, S588 and T589 (AAV6 VP1 numbering), and/or within the regions to be modified described; e.g. 262-272; 382-386, 445-457, 459, 469-473, 488-489, 494-496, 499-515, 571-579, 584-589, and 593-595 (AAV6 VP1 numbering). All combinations of amino acid substitutions. Briefly, for Gibson assembly, oligos with an average length of 70 nucleotides that contains at least 15-20 nt overlapping homology to the neighboring oligos will be generated. They will contain degenerate nucleotides (N K) within genomic amino acid regions coding for different modified regions. Plasmid libraries will generated by in vitro assembly of multiple oligos using High Fidelity Gibson Assembly Mix (NEB, Ipswich, Mass.) according to manufacturer instructions. The assembled fragments are either PCR amplified for 10 cycles using Phusion HF (NEB, Ipswich, Mass.) or directly cloned into pTR-AAV6 plasmids between the BspEI and Sbfl restriction sites. Plasmid pTR-AAV 6** contains genes encoding AAV2 Rep and AAV6 Cap with 2 stop codons at positions 490 and 491 (AAV6 VP1 numbering) introduced by site directed mutagenesis (Agilent, Santa Clara, Calif.). The entire construct is flanked by AAV2 inverted terminal repeats (ITRs) to enable packaging and replication of pseudotyped AAV 6 libraries upon helper virus co-infection. It is noteworthy to mention that the AAV6 capsid gene is incorporated prior to library cloning in order to reduce wild type AAV 6 contamination within the different libraries. Ligation reactions are then concentrated and purified by ethanol precipitation. Purified ligation products are electroporated into DH 10B electroMax (Invitrogen, Carlsbad, Calif.) and directly plated on multiple 5245 mm2 bioassay dishes (Corning, Corning, N.Y.) to avoid bias from bacterial suspension cultures. Plasmid DNA from pTR-AAV6 libraries is purified from pooled colonies grown on LB agar plates using a Maxiprep kit (Invitrogen, Carlsbad, Calif.).

Directed evolution of modified rAAV strains.

Equal amounts (15 ug each) of each pTR-AAV6 library and the Ad helper plasmid, pXX680, are transfected onto liver cells (or in some embodiments HEK293 cells) at 70-80% confluency on each 150 mm dish using PEI to generate the viral libraries with all combination. AAV libraries are purified using standard procedures. MB114 cells are seeded on a 100 mm tissue culture dish overnight to reach 60-70% confluence before inoculation with AAV libraries at an MOI ranging from 1000-10,000. After 24 h post-transduction, murine adenovirus. (ATCC) MAV-1 is added as helper virus to promote AAV replication. At 6 days post-infection with MAV-1 (50% CPE), the supernatant is harvested and DNase I resistant vector genomes quantified on day 7. Media containing replicating AAV strains and MAV-1 obtained from each round of infection are then used as inoculum for each subsequent cycle for a total of 5 rounds of evolution. Subsequent iterative rounds of evolution are carried out in a similar fashion with AAV capsid libraries containing different permutations and combinations of newly evolved antigenic footprints.

Identification of newly evolved AAV strains.

To analyze sequence diversity of the parental and evolved AAV6 saturation libraries, DNase I resistant vector genomes are isolated from media and amplified by Q5 polymerase for 10-18 cycles (NEB, Ipswich, Mass.) using primers, 5′-CCCTACACGACGCTCTTCCGATCT cagaactcaaaatcagtccggaagt-3′ (SEQ I D NO: 5) and 5′-GACTGGAGTTCAGACGTGTGCTCTTCCGATCTNNNNNgccaggtaatgctcccatagc-3′ (SEQ ID NO: 6). Illumina MiSeq sequencing adaptor for multiplexing was added through a second round of PCR using Q5 Polymerase with P5 and P7 primers. After each round of PCR, the products are purified using a PureLink PCR Micro Kit (ThermoFisher, Waltham, Mass.). Quality of the amplicons was verified using a Bioanalyzer (Agilent), and concentrations quantified using a Qubit spectrometer (ThermoFisher, Waltham, Mass.). Libraries are then prepared for sequencing with a MiSeq 300 Kit v2. following manufacturer instructions, and sequenced on the MiSeq system (Illumina).

Sequencing data analysis. De-multiplexed reads are analyzed via a custom Perl script. Briefly, raw sequencing files are probed for mutagenized regions of interest, and the frequencies of different nucleotide sequences in this region are counted and ranked for each library. Nucleotide sequences are also translated, and these amino acid sequences are similarly counted and ranked. Amino acid sequence frequencies across libraries were then plotted in R graphics package v3.2.4.

To characterize selected clones from each library, DNase I resistant vector genomes are isolated from media and amplified by Phusion I IF (NEB, Ipswich, Mass.) using primers flanking the BspEI and Sbfl sites. The PCR products are gel purified, sub-cloned into TOPO cloning vectors (ThermoFisher, Waltham, Mass.) and sent out for standard Sanger sequencing (Eton Bioscience, San Diego, Calif.). Unique sequences are sub-cloned into an AAV helper plasmid backbone, pXR, using BspEI and Sbfl sites. Unique recombinant AAV6 variants are produced following a standard rAAV production protocol as described above.

In vitro antibody and serum neutralization assays.

Twenty-five microliters of antibodies or antisera, e.g. ADK6 antibody or AAV1 neutralizing antibody, are mixed with an equal volume containing recombinant AAV6 vectors (MOI 1,000-10,000) in tissue culture treated, black, glass bottom 96 well plates (Corning, Corning, N.Y.) and incubated at room temperature (RT°) for 30 min. A total of 5×10⁴Liver cells (in some embodiments HEK293 cells) in 500 of media is then added to each well and the plates incubated in 5% CO2 at 37° C. for 48 h. Cells are then lysed with 25 μl of 1× passive lysis buffer (Promega, Madison, Wis.) for 30 min at RT. Luciferase activity is measured on a Victor 3 multilabel plate reader (Perkin Elmer, Waltham, Mass.) immediately after addition of 25 μl of luciferin (Promega, Madison, Wis.). All read outs are normalized to controls with no antibody/antisera treatment. Recombinant AAV vectors packaging ssCBA-Luc transgenes are pre diluted in DMEM+5% FBS+P/S are utilized in this assay.

In vivo antibody neutralization assay. Each hind limb of 6-8 week old female BAlb/c mice (Jackson Laboratory, Bay Harbor, Me.) is injected intramuscularly (I.M.) with 2×10¹⁰ AAV packaging CBA-Luc pre-mixed with different monoclonal antibodies, e.g. ADK6 and AAV1 neutralizing antibodies (e.g. 4E4, 5H7 and ADKla), at 1:500, 1:50 and 1:5 dilutions, in a final volume of 20 μi. After 4 wk post-injection, luciferase activity is measured using a Xenogen IVIS Lumina system (PerkinElmer Life Sciences/Caliper Life Sciences, Waltham, Mass.) at 5 min post-intraperitoneal (LP.) injection of 175 μi of in vivo D-luciferin (120 mg/kg Nanolight, Pinetop, Ariz.) per mouse. Luciferase activity is measured as photons/sec/cm2/sr and analyzed using Living Image 3.2 software (Caliper Life Sciences, Waltham, Mass.).

Generation of anti-AAV6 mouse serum by Immunization (AAV6 antisera). 1×10 vg of wtAAV6 in 20 μi of PBS is injected intramuscularly into each hind leg of 6-8 week old, female Balb/c mice. Whole blood is collected by cardiac puncture at 4 wk post-injection and serum was isolated using standard coagulation and centrifugation protocols. Briefly, mouse blood is coagulated at RT for 30 min and centrifuged at 2000 g for 10 min at 4° C. All serum is heat-inactivated at 55° C. for 15 min and stored at −80° C.

In vivo characterization of AAV variants in mice. A dose of 1×1 θ¹¹ vg of AAV vectors packaging the scCBh-GFP transgene cassette in 200 μl of PBS is injected into C57/B16 mice intravenously (l.V.) via the tail vein. Mice are sacrificed after 3 wk post-injection and perfused with 4% paraformaldehyde (PFA) in PBS. Multiple organs, including heart, brain, liver and kidney, are harvested. Tissues are sectioned to 50 μm thin slices by vibratome VT1200S (Leica, Welzlar, Germany) and stained for GFP with standard immunohistochemistry 3,3′-Diaminobenzidine (DAB) stain procedures described previously. At least 3 sections per organ from 3 different mice are submitted for slide scanning. For bio-distribution analysis, 1×10¹¹ vg of AAV vectors packaging ssCBA-Luc are injected 1.V. as mentioned above in Balb/C mice. After 2 wk post-injection, mice are sacrificed and perfused with 1 PBS. Multiple organs, including heart, brain, lung, liver, spleen, kidney and muscle, were harvested. DNA was harvested using DNeasy kit (Qiagen, Hilden, Germany) according to the manufacturer's instructions. Vector genome copy numbers are determined by quantitative PCR (qPCR) using as described previously using luciferase transgene primers, 5′-CCTTCGCTTC AAAAAATGGAAC-3′ (SEQ ID NO: 7), and 5′-AAAAGC ACTCTGATTGACAAATAC-3′ (SEQ ID NO: 8). Viral genome copy numbers are normalized to mouse genomic DNA in each sample. Tissue samples are also processed for luciferase activity assays by homogenization in 1×PLB (Promega, Madison, Wis.) using a Qiagen TissueLyserll at a frequency of 20 hz for three 45s pulses. The homogenate was spun down, and 20 μl of supernatant mixed with 500 of luciferin (Promega, Madison, Wis.) and immediately measured using a Victor 3 multilabel plate reader (Perkin Elmer, Waltham. Mass.).

It is expected that the saturated library described above, after going through 4-5 rounds of evolution will yield rAAV6 virions that retain their ability to bind to heparan sulfate and transduce the liver, yet uniquely have reduced neutralization by ADK6 antibodies. These modified vectors are also combined with modified amino acid regions known to have reduced neutralization to ADK1 and other cross reactive neutralizing antibodies and thus evade neutralization in vivo and in vitro while retaining liver transduction.

| SEQUENCES | |
|---|---|
| AAV6 capsid protein VP1 (GenBank Accession No. AAB95450) | |
| (SEQ ID NO: 1) | |
| 1 | MAADGYLPDW LEDNLSEGIR EWWDLKPGAP KPKANQQKQD DGRGLVLPGY KYLGPFNGLD |
| 61 | KGEPVNAADA AALEHDKAYD QQLKAGDNPY LRYNHADAEF QERLQEDTSF GGNLGRAVFQ |
| 121 | AKKRVLEPFG LVEEGA TAP GKKRPVEQSP QEPDSSSGIG KTGQQPAKKR LNFGQTGDSE |
| 181 | SVPDPQPLGE PPATPAAVGP TT ASGGGAP MADNNEGADG VGNASGNWHC DSTWLGDRVI |
| 241 | TTSTRTWALP TYNNHLYKQI SSASTGASND NHYFGYSTPW GYFDFNRFHC HFSPRDWQRL |
| 301 | INNNWGFRPK RLNF LFNIQ VKEVTTNDGV TTIANNLTST VQVFSDSEYQ LPYVLGSAHQ |
| 361 | GCLPPFPADV FMIPQYGYLT LNNGSQAVGR SSFYCLEYFP SQMLRTGNNF TFSYTFEDVP |
| 421 | FHSSYAHSQS LDRLMNPLID QYLYYLNRTQ NQSGSAQNKD LLFSRGSPAG MSVQPKNWLP |
| 481 | GPCYRQQRVS KTKTDNNNSN FTWTGASKYN LNGRESIINP GTAMASHKDD KDKFFPMSGV |
| 541 | MIFG ESAGA SNTALDNVMI TDEEEIKATN PVAIERFGTV AVNLQSSSTD PATGDVHVMG |
| 601 | ALPGMVWQDR DVYLQGPIWA KIPHTDGHFH PSPLMGGFGL KHPPPQILIK NTPVPANPPA |
| 661 | EFSATKFASF ITQYSTGQVS VEIEWELQKE NSKRWNPEVQ YTSNYAKSAN VDFTVDNNGL |
| 721 | YTEPRPIGTR YLTRPL |

TABLE 1
Antibodies specific for AAV6

| Target | Clone Name | Reference Information |
|---|---|---|
| AAV6 | 10R-2480 | FITZGERALD INDUSTRIES |
| | | INTNL MS |
| AAV6 | ADK6 | Progen |

We claim:

1. A modified recombinant AAV6 vector comprising an amino acid substitution at one or more amino acid residues selected from the group consisting of 5264, G266, N269, H272, Q457, S588 and T589 corresponding to AAV6 VP1 numbering, wherein the rAAV6 vector transduces the liver and has reduced neutralization of transduction of liver by ADK6 antibody as compared to the rAAV6 vector lacking the one or more substitutions.

2. The modified AAV6 vector of claim 1, further comprising a lysine (K) or arginine (R) at an amino acid site corresponding to amino acid 531 of AAV6 VP1.

3. The modified AAV6 vector of claim 2 comprising a K at amino acid 531.

4. The modified AAV6 vector of claim 2, comprising a R at amino acid 531.

5. The modified AAV6 vector of any of claims 1-3, wherein at least two of the one or more amino acids are substituted.

6. The modified AAV6 vector of any of claims 1-4, wherein at least three of the one or more amino acids are substituted.

7. The modified AAV6 vector of any of claims 1-5, wherein at least four of the one or more amino acids are substituted.

8. The modified AAV6 vector of any of claims 1-6, wherein at least five of the one or more amino acids are substituted.

9. The modified AAV6 vector of any of claims 1-7, wherein at least six of the one or more amino acids are substituted.

10. The modified AAV6 vector of any of claims 1-8, wherein at least seven of the one or more amino acids are substituted.

11. The modified AAV6 vector of any of claims 1-9 wherein the one or more substitutions comprise conserved substitutions.

12. The modified AAV6 vector of any of claims 1-9 wherein the one or more substitutions comprise non-conserved substitutions.

13. The modified rAAV6 vector of any of claims 1-11, further comprising a substitution of at least one amino acids that binds to sialic acid selected form the group consisting of: N447, 5472, V473, N500, T502, and W503 corresponding to AAV6 VP1 numbering.

14. The modified rAAV6 vector of any of claims 1-12, further comprising one or more modified regions of amino acids selected from the group consisting of: 262-272; 382-386, 445-457, 459, 469-473, 488-489, 494-496, 499-515, 571-579, 584-589, and 593-595, corresponding to AAV6 VP1 numbering, and wherein at least one or more amino acids in the one or more modified regions are substituted.

15. A modified recombinant AAV6 vector comprising a substitution at one or more amino acid residues selected from the group consisting of S264, G266, N269, H272, Q457, S588 and T589 corresponding to AAV6 VP1 numbering, wherein the rAAV6 vector transduces the liver and has reduced neutralization of transduction by ADK6 antibody as compared to the rAAV6 vector lacking the one or more substitutions.

16. A modified rAAV6 vector comprising one or more modified regions of amino acids selected from the group consisting of: 262-272; 382-386, 445-457, 459, 469-473, 488-489, 494-496, 499-515, 571-579, 584-589, and 593-595 corresponding to AAV6 VP1 numbering wherein at least one or more amino acids in the one or more modified regions are substituted, and wherein the rAAV6 vector transduces the liver and has reduced neutralization of transduction of liver by ADK6 antibody as compared to the rAAV6 vector lacking the one or more substitutions.

17. The modified rAAV6 vector of claim 15 comprising K531 corresponding to AAV6 VP1 numbering.

18. The modified rAAV6 vector of claim 15 comprising R531 corresponding to AAV6 VP1 numbering.

19. The modified rAAV6 vector of any of claims 1-17 further comprising amino acid substitution at one or more amino acid regions selected from the group consisting of: 456-459;
492-499; and 588-597.

20. The modified rAAV6 vector of any of claims 1-18, comprising one or more of the amino acid sequences selected from the group consisting of: SEER at 456-499 (SEQ ID NO: 2), TPGGNATR (SEQ ID NO: 3) at 492-499; DLDPKATEVE (SEQ ID NO: 4) at 588-597.

21. The modified rAAV6 vector of any of claims 1-19, wherein the vector has reduced neutralization of liver transduction by human anti-sera to unmodified rAAV6 virus as compared to neutralization of the unmodified rAAV6 vector.

22. The modified rAAV6 vector of any of claims 1-19, wherein the vector has reduced neutralization of liver transduction as measured by mouse anti-sera to unmodified rAAV6 virus as compared to neutralization of the unmodified rAAV6 vector.

23. The modified rAAV6 vector of any of claims 1-19, wherein the vector has reduced neutralization of liver transduction as measured by rhesus macaques anti-sera to unmodified rAAV6 virus as compared to neutralization of the unmodified rAAV6 vector.

24. A method for identifying an AAV6 virion that retains liver tropism and exhibits reduced neutralization by ADK6 antibody comprising:
a. generating a saturation mutagenesis AAV6 library wherein each of the amino acids selected from the group consisting of 5264, G266, N269, H272, Q457, 5588 and T589, are substituted with each of the 20 different natural or unnatural amino acid and in any combination of all or fewer than all positions, and wherein the AAV6 comprises either K531 or R531; and
b. performing rounds of evolution by infecting liver cells (e.g. hepatocytes) or tissue with the rAAV6 library; and
c. screening for reduced neutralization by of at least 10% ADK6 or anti-sera as compared to the corresponding unmodified AAV6 virion.

25. The method of claim 23, further comprising screening for the loss of sialic acid binding.

26. The method of claim 23, further comprising screening for the presence of sialic acid binding.

27. A method for identifying an AAV6 virion that retains liver tropism and exhibits reduced neutralization by ADK6 antibody comprising:
a. generating a saturation mutagenesis library of one or more modified regions of amino acids selected from the group consisting of: 262-272; 382-386, 445-457, 459, 469-473, 488-489, 494-496, 499-515, 571-579, 584-589, and 593-595, wherein the one or more regions are substituted with each of the 20 different natural or unnatural amino acid and in any combination of all or fewer than all positions, and wherein the AAV6 comprises either K531 or R531; and
b. performing rounds of evolution by infecting liver cells (e.g. hepatocytes) or tissue with the rAAV6 library; and
c. screening for reduced neutralization of at least 10% by ADK6 or anti-sera as compared to the corresponding unmodified AAV6 virion.

28. The method of claim 27, further comprising screening for the loss of sialic acid binding.

29. The method of claim 27, further comprising screening for the presence of sialic acid binding.

30. A modified rAAV6 vector obtained by the method of any of claims 24-27, wherein the modified rAAV6 vector comprises reduced neutralization by ADK6 antibody and transduces the liver.

31. The modified rAAV6 vector of claim 28, wherein the vector has at least 10% reduced neutralization of liver transduction by human anti-sera to unmodified rAAV6 virus, as compared to the neutralization of unmodified rAAV6 vector.

32. The modified rAAV6 vector of claim 28, wherein the vector has at least 10% reduced neutralization of liver transduction by mouse anti-sera to unmodified rAAV6 virus, as compared to the neutralization of unmodified rAAV6 vector.

33. The modified rAAV6 vector of claim 28, wherein the vector has at least 10% reduced neutralization of liver transduction by rhesus macaques anti-sera to unmodified rAAV6 virus, as compared to neutralization of the unmodified rAAV6 vector.
